(19) United States
(12) Patent Application Publication
(10) Pub. No.: US 2026/0020883 A1
(43) Pub. Date: Jan. 22, 2026

(54) LOCKING MODULAR PLATING DEVICES, METHODS AND SYSTEMS

(71) Applicants: Amazing Brace LLC, Tampa, FL (US); Misty Mountain Spine Inc., Sherman Oaks, CA (US)

(72) Inventors: Dimitriy KONDRASHOV, Sherman Oaks, CA (US); Victor Hayes, Tampa, FL (US)

(21) Appl. No.: 19/271,756

(22) Filed: Jul. 16, 2025

Related U.S. Application Data
(60) Provisional application No. 63/672,218, filed on Jul. 16, 2024.

(51) Int. Cl. A61B 17/70; A61B 17/17; A61B 17/80

(52) U.S. Cl. CPC A61B 17/7058; A61B 17/1728; A61B 17/1757; A61B 17/809

(57) ABSTRACT

Provided are bone plates and systems including such plates. The present plates may be used for example, to accommodate spinolaminar junction or as a plate locking screw system for posterior spinal fixation in the cervical, thoracic, lumbar and sacral spine, extremities, pelvis and long bone fixation. The present devices and systems may include modular plates, rods and rails. Devices and systems herein relate to both open methods and minimally invasive surgery applications. Also provided are locking plates and systems relating to tethering applications. Further provided are kits including any of the present kits or devices, and methods including insertion of any of the present plates or systems into a patient.

### CROSS REFERENCE TO RELATED APPLICATION

This application claims the benefit of U.S. Provisional Application 63/672,218 filed on Jul. 16, 2024, the contents of which are hereby incorporated herein by reference in their entirety.

### FIELD

Embodiments of the present application generally relate to bone plates, such as locking plates and other devices, systems and methods to stabilize a spinal column including for posterior spinal fixation in the cervical, thoracic, lumbar and sacral spine, extremities, pelvis and long bones. Embodiments also relate to accessory spine instruments to assist in reduction of spine instability or spinal deformity. Embodiments also relate to methods of using the present bone plates and systems, such as spinolaminar plates, for example to revise a loose pedicle screw in a patient, without removing an old construct in the patient.

### BACKGROUND

It can be desirable to correct spine deformity, address spine instability or reduce the compression of spinal nerves/spinal cord by stabilizing the spinal column. The spinal column can be stabilized by isolating spinal motion segment(s) and restricting relative motion between adjacent vertebrae. This may be difficult, however, in patients with low bone density, which compromises screw purchase, screw push-in torque and screw pullout strength. It also can be very challenging in patients with significant loss of bony landmarks for example in cases of prior surgery and significant spinal deformity.

### SUMMARY

Provided herein are bone plates (such as locking plates) and systems, for open and minimally invasive surgery such as spinal fixation surgery (e.g. posterior spinal fixation), and for spinal, hib, extremity and long bone fixation systems including modular plates, rods and rails; and methods that use bone plates for example, to revise a loose pedicle screw without removing existing/old constructs in a patient. Also provided are locking plates and systems that may be adapted for cervical and sacral spine applications. Also provided are locking plates and systems relating to spine tethering applications. Further provided are other methods including the present plates and systems.

Provided are bone plates that include a main body having a plurality of screw openings, in which the main body is configured in shape and/or size to generally correspond to bone of a patient to which the bone plate is to be applied, and in which a screw opening of the plurality of screw openings has threading configured to accept a respective screw or screw-in attachment therein.

Also provided are bone plate systems that include a bone plate provided herein and at least one screw or screw-in attachment corresponding to a respective screw opening of the plurality of screw openings in the bone plate.

Also provided are kits that include a bone plate provided herein and at least one additional component including instructions for use or insertion of the bone plate or system, one or more screws, screw-in attachments, rods, plates, rails, bars, offset connectors, drill guides, guide wires, screwdrivers, counter torque, retaining pins, wires drills and other tools for insertion, placement, attachment or visualization of the device(s) or system(s) into a patient.

Further kits provided herein may include multiple bone plates of varying sizes and/or shapes. In example embodiments, each bone plate of the multiple bone plates may include a main body having a plurality of screw openings, in which the main body is configured in shape to correspond to bone of a patient, and in which at least one screw opening of the plurality of screw openings has threading configured to accept a respective screw or screw-in attachment therein.

Also provided are methods that include placing a bone plate at a desired location in a patient in need of the bone plate, for example for stabilization or fixation, and securing the bone plate to bone of the patient by screwing at least one screw through a corresponding screw opening of the bone plate, into the bone of the patient. In example embodiments, the bone plate includes a main body having a plurality of screw openings therein, the main body of the bone plate is shaped to correspond to bone of the patient at the desired location to which the bone plate is to be applied, and the at least one screw opening of the plurality of screw openings has threading configured to accept a respective screw or screw-in attachment therein.

Also provided are methods that include placing a bone plate system having a bone plate and at least one drill guide, at a desired location in a patient in need thereof, creating a pilot hole in the bone of the patient through at least one drill guide, removing the drill guide from the bone plate system, and screwing a screw into the pilot hole in the bone of the patient through the bone plate to attach the bone plate to the bone of the patient. In example embodiments, the bone plate includes a main body having a plurality of screw openings therein, in which the main body is shaped to correspond to bone of the patient to which the bone plate is to be applied, and at least one screw opening of the plurality of screw openings has threading configured to accept the screw from the at least one screw therein. In example embodiments, the at least one drill guide is positioned on the bone plate at a corresponding screw opening of the plurality of screw openings.

Further methods provided herein include
a) determining a size of a lamina of a patient,
b) selecting locking plate of a corresponding size to be applied to the patient, based on the determined size of the lamina,
c) attaching a plate holder into a preloaded drill guide on the selected locking plate,
d) placing the locking plate over the lamina of the patient,
e) creating a first pilot hole in the lamina of the patient through a first hole in the locking plate using a first preloaded drill guide corresponding to the first hole in the locking plate,
f) screwing a first locking screw in the first pilot hole in the lamina of the patient through the first hole in the locking plate,
g) repeating steps e) and f) at least one time, drilling a second or more pilot hole through a second or more hole in the locking plate, and screwing a second or more locking screw in a corresponding pilot hole in the lamina of the patient through a corresponding hole in the locking plate, and
h) removing the plate holder from the patient.

Also provided are patient-specific spinolaminar locking plates and fixation systems for segmental fixation, for example, during spine fusion surgery. Such methods include measuring and/or visualizing regional bone density in a patient, prior to selecting a suitable bone plate from the present bone plates and bone plate systems. These methods may be used for example for spinolaminar locking plate segmental fixation of the spine.

### DESCRIPTION OF THE DRAWINGS

Non-limiting example embodiments described herein, with reference to the following accompanying Figures.

FIGS. 1A-1D depict a bone plate in accordance with non-limiting embodiments of the present application, from different angles.

FIGS. 2A-2D depict a bone plate system that includes a bone plate with a screw inserted in each screw hole of the bone plate, in accordance with non-limiting embodiments, from different angles.

FIGS. 3A-3D depict a bone plate system that includes a bone plate having a guide corresponding to each screw hole of the bone plate, in accordance with non-limiting embodiments, from different angles.

FIGS. 4A-4D depict example locking screws with threaded heads to engage threads on a plate, in accordance with non-limiting embodiments.

FIGS. 5A-5D depict example non-locking lag screws that may be used with the present bone plates, in accordance with non-limiting embodiments.

FIG. 6 depicts an example of a lag screw in an example bone plate system of the present application.

FIG. 7 depicts example screws that may be used in accordance with the present application. Depicted are a combination of locking and non-locking screws and some tulip screws, some fully threaded, some partially threaded and some unthreaded.

FIG. 8 depicts example screws that may be used in accordance with the present application, including optional sizes, such as diameter sizes, of the screws.

FIG. 9 depicts a combination of locking and non-locking screws that may be used in accordance with the present application.

FIG. 10 depicts both locking and non-locking screws of various diameters in accordance with the present application.

FIGS. 11A-11D depict monoaxial tulip screws that may be used in the present systems according to example embodiments of the present application, with a threaded head portion to engage the plate.

FIGS. 12A-12D depict polyaxial tulip screws that may be used in the present systems according to example embodiments of the present application, with a threaded head portion to engage the plate.

FIGS. 13A-13D depict an outrigger bone plate including multiple diverging and converging (non-parallel and/or parallel) screw holes in accordance with example embodiments.

FIGS. 14A-14B depict an example of a precontoured 6-hole locking plate with threaded holes in accordance with example embodiments.

FIGS. 15-18 depict examples of the placement of different spinal bone plates, having different configurations and some with guides and some without guides, at adjacent segments of the spine.

FIGS. 19A-19D depict an example of a 4-hole bone plate from four views, with divergent screws in holes of the bone plate, in different planes.

FIGS. 19E-19I depict a transparent rendering of an example of a 4-hole bone plate from five views, with divergent screws in holes of the bone plate.

FIG. 20 depicts a plate having screw holes and an eyelet screw-in accessory that may be screwed into a screw hole, for a sublaminar band, wire, suture or cable passage.

FIG. 21 depicts a plate with another example embodiment of an eyelet with locking threads, which allows for both locking into the plate as well as passage of the sublaminar band. Locking threads can be engaged in any of the locking holes in the spinal laminar plate.

FIG. 22 depicts a blocking plate with a tulip on the top of the blocking plate. The blocking plate may be attached to the top of a bone plate in example embodiments. Also shown is a screw in an eyelet that may be screwed into a screw hole of a bone plate, which allows for cable, suture or band passage. Further shown are spikes for initial/provisional fixation of the plate.

FIG. 23 depicts a bone plate according to non-limiting example embodiments that has pre-loaded drill guides. Example plates may also have spikes/protruding surface features on an opposite side from the pre-loaded drill guides to provide initial stability of the bone plate on bone in a patient.

FIG. 24 depicts a hybrid construct using example bone plates along with a pedicle screw used at the same level and same side as the bone plate with plate.

FIG. 25 depicts a gradient of stiffness formed in a patient. A spinolaminar plate according to example embodiments, can accommodate one, two, three or more tulips that can accept rods of different material and diameter.

FIG. 26 depicts a three-hole plate for pedicle screw augmentation according to non-limiting example embodiments. The example bone plate may have one, two, three or more locking, non-locking or hybrid holes that may be configured to accommodate one or more screws, and/or one or more screw-in attachments, such as the example depicted tulip screw, which may be adapted to attach to a rod or connector. The plate is connected to a preexisting screw/rod construct via a lateralizing, offset or iliac-type connector.

FIG. 27 relates to tethering applications in which a locking plate can also be applied to the vertebral body or posterior elements either anteriorly, laterally or anterolaterally. The tulip in this application may accept a flexible non-rigid tether such as a braided polymer to allow for motion preservation.

FIG. 28 depicts another example of a hybrid construct incorporating plates and pedicle screws in the same construct.

FIG. 29 depicts a mini hook plate in accordance with example embodiments. The hook engages lamina and improves pullout strength.

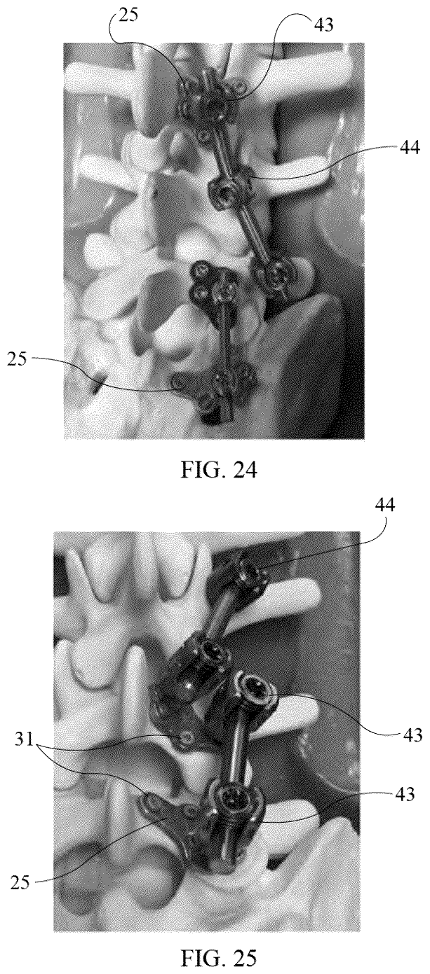

FIG. 30 depicts a mini version of example bone plates in accordance with example embodiments, which may be used after laminectomy/Transforaminal Lumbar Interbody Fusion (TLIF) cage placement. Example embodiments may have a limited number of holes and smaller footprint than other example plates. FIG. 30 depicts an example spinolaminar plate that can be adapted for minimally invasive surgery (MIS) insertion into a patient.

FIG. 31 depicts example embodiments of bone plates that include a locking cannulated screw or cap with an inner hollow channel that can accommodate bands (such as FiberWire or any other synthetic material suture, tape or bands) for additional spinal fixation. The locking screw can be passed for example through an inside channel in the screw. This can be used to augment soft tissue or bone repair. FIG. 31 depicts an example locking screw that can be used with present bone plates that can have a cannulation to allow for passage of suture.

FIG. 32 depicts an example bone plate that may have fixed tulips on its surface to provide rigidity.

FIG. 33 depicts example embodiments having a monoaxial locking screw. The screw may have a fixed angle or an angle tulip that may be configured for receiving a rod or bar or rail therein. The said screw has a threaded head to engage the plate.

According to non-limiting example embodiments, a modular plate/rod/rail fixation system may be used for extremity fixation, such as in fixation of long bone fractures.

FIG. 34 depicts example bone plates applied to a long bone, the bone plates having attachments to engage with rods, bars or rails. This example system may be used for example to span a fracture in the long bone.

FIG. 35 depicts example bone plates that may be used for long bone fracture fixed with dual construct of modular locking plates with one, two, three or more rods, bars or rails spanning the fracture.

FIG. 36 depicts an example modular locking plate for extremity fracture fixation. A locking set screw can lock a circular, elliptical, rectangular, square or differently shaped bar or rod or rail depending on surgeon's preference and the rigidity of the fracture fixation desired. Such plate can be placed through a small incision and the rod, bar or rail can be passed under the muscle or fascia to minimize soft tissue trauma.

In example embodiments, segmental locking plates with polyaxial or monoaxial saddles (tulips) may be used for internal fixation of extremity fracture. One, two or plurality of the plates with one, two or plurality of locking screws may be fitted with one, two or plurality of poly- or monoaxial tulips on either side of the fracture, The individual plates may be connected with one, two or plurality of rods, bars or rails or combinations of such.

FIG. 37 depicts an example linear locking plate that may be used for non-spinal (e.g., rib or pelvic) fixation according to non-limiting example embodiments.

FIG. 38 depicts a Densitometry Map used to measure regional bone density, which may be used according to non-limiting example embodiments of the present application in selecting patient-specific method of spinolaminar locking plate segmental fixation of the spine.

FIG. 39 is an example of multilevel cervical plate construct in accordance with non-limiting examples.

FIG. 40 depicts a hybrid cervicothoracic plate/pedicle screw construct.

FIGS. 41A and 41B depict an example of a drill guide from different perspective views.

FIGS. 42A-42C depict a whole system in accordance with non-limiting example embodiments including multiple bone plates, screws, and tulip screws fixing the bone plates to the spine of a patient, and fixing the bone plate to a rod system to provide supplemental fixation.

### DETAILED DESCRIPTION

Example embodiments provided herein include bone plates and systems including such plates, kits including the present bone plates and open and minimally invasive methods for the placement and use of bone plates in a patient.

Spine surgeons may use bone plates or locking plates as an alternative to pedicle screws to help patients, for example, with spinal fusion surgery regardless of their bone condition and the extent of their spinal deformity. Non-limiting examples of spinolaminar locking plates are provided for example in U.S. Pat. No. 8,623,062 issued on Jan. 7, 2014, which is incorporated herein by reference in its entirety. Locking plates may be segmental, meaning that it is only fixed to one vertebra and do not bridge several vertebrae together. However, in case of cervical spine, the plates maybe bridging two or more segments together. The present application includes for example, use of locking plates that are designed to fixate, e.g., on the densest regions of the vertebrae and gives the surgeon a safer and potentially stronger spinal anchor. Example embodiments may include a plate, a plurality of locking screws, a tulip assembly (monoaxial, preferred angle, uniplanar or polyaxial), a set screw and a rod/rail assembly. The plurality of locking and non-locking screws allows the present systems and methods to securely attach to bone of the patients, such as spinal bone without the need of engaging the full length of the pedicles and the vertebral body. The locking screws may be placing unicortically, without engaging the dar cortex of the bone. They also may be placed in a diverging and/or converging configuration to widen the footprint and optimize pullout strength of the plate. Thus, the present application includes a bone plate and screw system that provides patients of all ages and conditions with a secure bone attachment, such as a spinal attachment over a larger footprint ensuring the fixation is safer and casier during the spinal fusion surgery. In an embodiment of the application, a spinolaminar locking plate includes a pre-contoured anatomical plate utilizing locking screws for posterior stabilization of the thoracolumbar, cervical or sacral spine, as well as occiput and pelvis. The disclosed systems and methods of the present application may also be used with systems and methods to restrict or eliminate relative motion of vertebral segments.

The present plates and systems may also be used for example for extremity plating, in addition to spine plating. Accordingly, methods herein may include methods of using the present plates in surgical methods relating to the spine or outside the spine, such as for extremities, pelvic bone or ribs. The present plates can also be used for sacral/pelvic fixation.

The present novel plates, systems, and methods are safer and less complicated to place than a pedical screw. The present system is simpler and less risky to use than pedicle screw system and does not require complex navigation or other systems to appropriately place, mainly because the locking screws are fixated in cortical bone posteriorly away from the neural, vascular and visceral structures. To work appropriately the locking screws in the present novel system are not required to pass through the pedicles nor the vertebral body nor be bicortical. Avoiding the pedicle reduces risk or breech, fracture and/or damage to the vital surrounding structures. When the locking screws are placed they will not penetrate deep enough to enter the spinal canal nor areas where neural/vascular structures are located. this reduces risk of implantation significantly.

Examples of the present bone plate (also referred to herein as a locking plate) system provides a unique way of instrumenting the posterior spine in the cervical thoracic and lumbar regions without the use of pedicle screws, wires, or bands.

Embodiments of the present locking plate system can be used to supplement standard pedicle screw systems to strengthen the construct in patients with osteoporosis or dysplastic bone.

While embodiments are described herein, it is to be understood that this disclosure is illustrative and exemplary. The detailed disclosure herein of example embodiments is not intended, nor is to be construed, to be limiting. All illustrations of the figures are for the purpose of describing selected embodiments and are not intended to limit the scope of the present application.

As should be understood, any embodiment may incorporate only one or a plurality of the above-disclosed aspects of the disclosure and may further incorporate only one or a plurality of the above-disclosed features. Items described in the singular herein may be provided in plural, as can be seen, for example, in the drawings. Thus, the description of a single item that is provided in plural should be understood to be applicable to the remaining plurality of items unless context indicates otherwise.

Each term used herein refers to that which an ordinary artisan would understand such term to mean based on the contextual use of such term herein. To the extent the meaning of a term used herein—as understood by the ordinary artisan differs in any way from any particular dictionary definition of such term, the meaning of the term as understood by the ordinary artisan in the context of the present application, should prevail.

The terms “a” or “an”, as used herein, are defined as one or more than one unless the contextual use dictates otherwise. The term plurality, as used herein, is defined as two or more than two. The term another, as used herein, is defined as at least a second or more.

When used herein to join a list of items, “or” denotes “at least one of the items,” but does not exclude a plurality of items of the list.

Throughout the specification, when a component is described as “including” a particular element or group of elements, it is to be understood that the component is formed of only the element or the group of elements, or the element or group of elements may be combined with additional elements to form the component, unless the context indicates otherwise.

It will be understood that when an element is referred to as being “connected” or “coupled” to or “on” another element, it can be directly connected or coupled to or on the other element or intervening elements may be present.

Ordinal numbers such as “first,” “second,” “third,” “primary,” “secondary” etc. may be used simply as labels of certain elements, steps, etc., to distinguish such elements, steps, etc. from one another. Terms that are not described using “first,” “second,” etc., in the specification, may still be referred to as “first” or “second” in a claim. In addition, a term that is referenced with a particular ordinal number (e.g., “first” in a particular claim) may be referenced elsewhere without an ordinal number or with a different ordinal number (e.g., “second” in the specification or another claim).

Any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Spatially relative terms, such as “below,” “lower,” “above,” “upper,” “top,” “bottom,” and the like, may be used herein for ease of description to describe positional relationships, such as illustrated in the figures, for example. It will be understood that the spatially relative terms encompass different orientations of the device in addition to the orientation depicted in the figures.

The following detailed description refers to the accompanying drawings. Wherever possible, the same reference numbers are used in the drawings and the following description to refer to the same or similar elements. While embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure.

### Bone Plates

Provided are bone plates (also referred to herein as locking plates) that include a main body having a plurality of screw openings, in which the main body is configured in shape and/or size to generally or approximately correspond to bone of a patient to which the bone plate is to be applied, and in which at least one screw opening of the plurality of screw openings has threading configured to accept a respective screw or screw-in attachment therein.

In example embodiments, the threaded portion(s) of the screw hole(s) may have multiple purposes: it may act to lock a screw to the plate, as a screw in place for the drill guides, and it also functions as a screw in for the plate holder during installation.

When the word “correspond” is used herein, it is used to mean that the general shape and size of the bone plate or main body of the bone plate conforms to the size or shape of the location to which it is applied. It is not intended to require an exact fit with contact on the entire surface of the bone plate to the bone of a patient, although exact conformation is included as well.

In stating that the main body is configured to correspond to bone of a patient, embodiments of bone plates systems and methods in accordance with the present application may include for example an anatomically contoured plate for posterior fixation of the thoracolumbar spine.

Example anchoring plates in accordance with the present application, may have a shape that is generally conformal with the outer surface of the targeted vertebra and generally spans the surface of the lamina. The shape of the anchoring plate further allows the anchoring plate to be secured to the structures so that a load applied to the anchoring plate during extension motion and flexion motion of the spinal column is distributed across the multiple structures to reduce a concentration of compressive or tensile force. Reducing compressive or tensile force can be particularly useful where the bone of one or more of the structures has degenerated, deteriorated or otherwise been weakened, for example in osteoporotic/osteopenic or previously operated patients.

According to example embodiments, the at least one screw opening of the plurality of screw openings is at an oblique angle with respect to a surface of the main body, such that a screw or screw-in attachment inserted into the at least one screw opening is at an oblique angle with respect to the surface of the main body.

According to example embodiments, screw openings may be oblique with respect to each other, and not necessarily with respect to the plate. In such embodiments, any screws or screw-in attachments inserted in the screw openings will be oblique with respect to each other. This may result in diverging and/or converging screws. In example embodiments the present application includes a locking plate system with converging and diverging screws to obtain supplemental fixation and standalone fixation in the posterior elements/structures of the cervical thoracic and lumbar spine. Multiple converging in diverging, locking screws increase the pull out strength of the system when compared to a single pedicle screws. The locked plate system may provide one solid point of fixation with converging and/or diverging screws to increase pullout strength and prevent loosening.

Example provided herein include bone plates in which the first screw opening of the plurality of screw openings is at an oblique angle with respect to a second screw opening of the plurality of screw openings, such that a first screw or screw-in attachment inserted into the first screw opening is at an oblique angle with respect to a second screw or screw in attachment inserted into the second screw opening.

According to other non-limiting example embodiments, bone plates may be provided in which the screw openings are not necessarily at oblique angles with respect to one another or with respect to a surface of the bone plate.

The present bone plates with e.g., locking screws do not need to be long or placed bicortically because the locking plate with diverging/converging screws create a fixed-angle construct which does not depend on bicortical screw purchase and has been used successfully on osteoporotic extremity fracture fixation.

The term “screw” is intended to encompass for example any type of screw that may be inserted into a screw opening of a plate of the present application. Numerous screws are contemplated including for example a lag screw, a lock screw, and a tulip screw, but are not limited to these screws. By way of example, a tulip screw may include a monoaxial tulip screw or a polyaxial tulip screw.

The screw may have a top surface on a same or similar plane to the top surface of the bone plate, or it may protrude above the top surface of the bone plate, or it may have an additional component above the top surface of the bone plate, for example in a case of a tulip screw.

The term “screw-in attachment” is intended to encompass any attachment that may be screwed into the screw holes of the plates-such as e.g. a screw-in eyelet, a suture band anchor, or other attachments that may be advantageous to surgical methods involving a fixation to bone.

In example embodiments some plates can be used without lag screw(s). However, all the plates will have at least one locking screw with an optional hole or screw hole for a connector or other screw-in attachment, which threads into the locking plate securely. This connector can be a blunt or screw-in tulip, eyelet screw, screw in crosslink or other configuration. This screw-in connector or attachment functions as an attachment to interconnect the plate to other segmental spine fixation systems and/or or another plates.

In example embodiments, a tulip may accept a rod connector, or component to accept a rod and/or crossconnector which secures the plate to another non-segmental or segmental system.

Example bone plates may be symmetrical or asymmetrical.

Example bone plates may be flat, curved, bent or may have portions that include one or more of these configurations depending on where the bone plate is to be applied and for which purpose.

According to non-limiting examples, the main body 2 may be bent at a central portion and include for example a first side 3 (which should not be considered to be limited in size or shape), and a second side 4 at an offset angle with respect to the second side. For example, the first and second side may be at an angle Θ of greater than 0 degrees to less than 360 degrees with respect to each other, or greater than 0 to less than 180 degrees. According to further non-limiting embodiments, the plates may vary in angle from 90 to 140 degrees or from 100 to 125 degrees. The angle may be determined or selected based for example on the anatomy of the location to which the plate is to be applied.

In non-limiting examples, a first side 3 of example embodiments may include for example, at least one of, a pair or more than two screw openings 5 which have threading 7 to be able to receive a screw 8 therein, and a second side 4 may include at least one, a pair, or more than two screw openings 5.

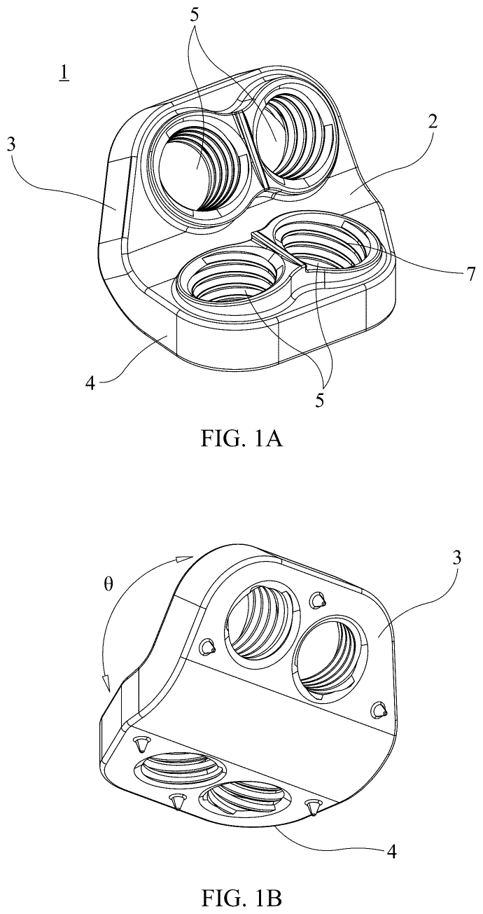

In example embodiments, the first side and the second side may be symmetrical with respect to each other as shown for example in FIGS. 1A-1D.

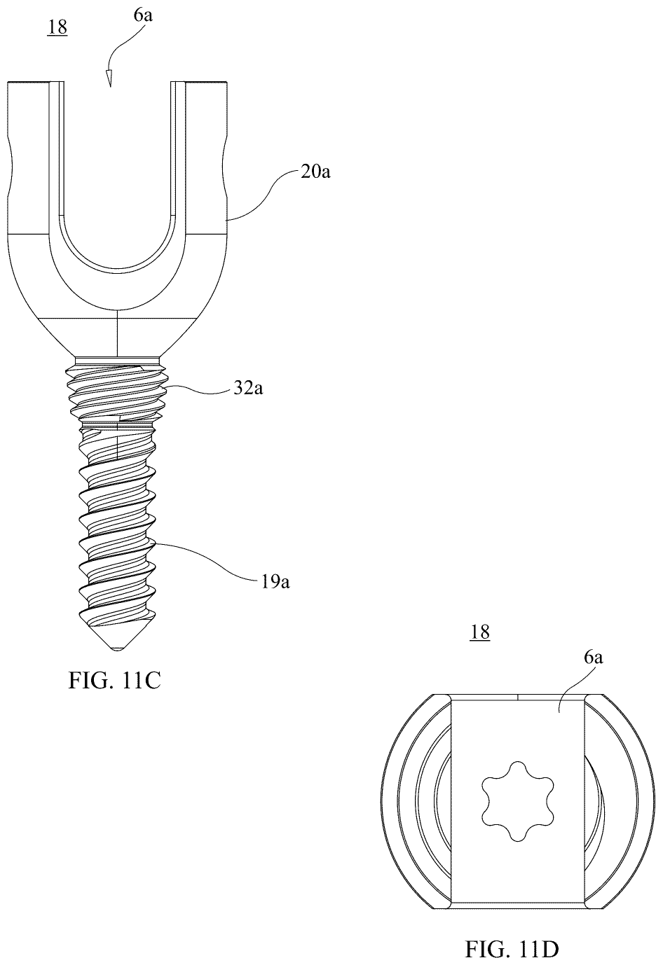

As can be seen for example in FIG. 1A and FIG. 1C, in example embodiments, the bone plate may be thicker in a diameter surrounding a circumference of the screw holes. This increased plate thickness surrounds the screw holes and has locking threads on the internal surface, which function to engage a guide and to lock the screws, tulip screws or screw-in attachments into the plate. As can be seen for example in FIGS. 13A and 13C, the plate may be thicker 66 around the screw holes and there may be threads inside the elevated part of the place, which threads continue into the plate. This raised border of plate thickness surrounding the screw holes adds a few extra threads. which improves the ease of placing the drill guides and improves the locking between the screwhead and plate. This feature also minimizes cross-threading. Some existing extremity locking plates are plagued by difficulty engaging the drill guides, which this aspect of example embodiments, addresses. In example embodiments, the thickness of the screw hole surrounding area may be uniform or it may be asymmetrical, depending for example on the trajectory/angle of the screw hole. For example, the plate diameter may be thicker around an upper portion of the angle of the hole and thinner (although still thicker than the plate itself) on a bottom side of the angled hole. The height and width of the extra thickness circumferentially present around screw holes, may be millimeters wide and thick.

Although described with respect to FIGS. 1A, 1C, 13A and 13C, it should be understood that bone plates having an increased thickness around a corresponding screw hole, and having threading within an inner circumference of the area of extra thickness, may be present with respect to any of the screw holes in any of the embodiments herein.

The area(s) of increased thickness around a corresponding screw hole may be the same or different material than that of the bone plate and may be made concurrently with making the bone plate or may be added to the bone plate thereafter, for example by being coated onto the bone plate or attached thereto, for example by glue.

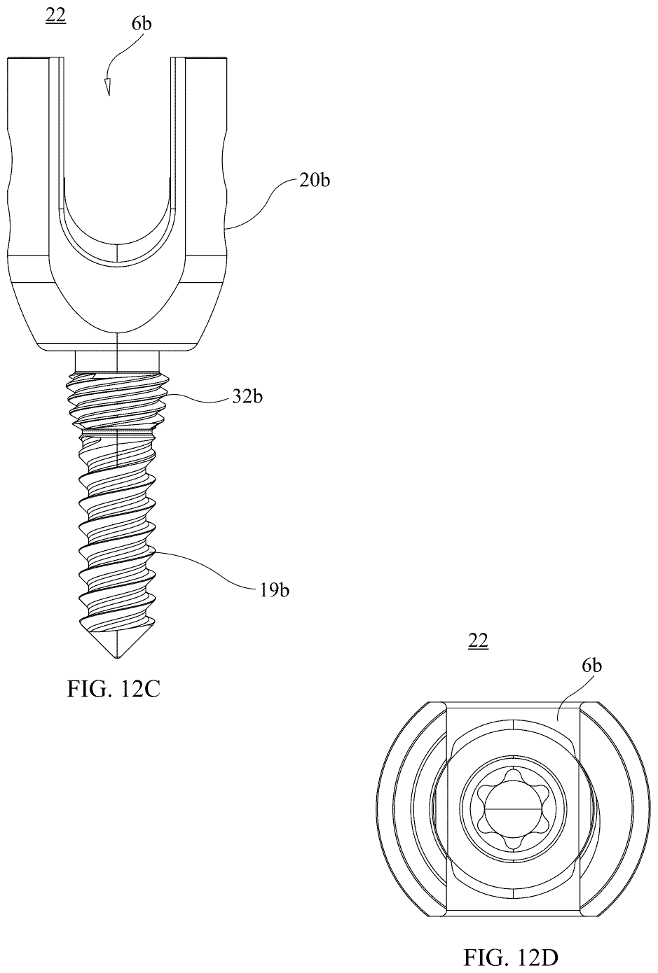

In other example embodiments, the first side and the second side may be offset with respect to each other as shown for example in FIG. 15, which depicts bend offset connectors.

FIGS. 1A-1D depict a bone plate in accordance with non-limiting embodiments of the present application, from different angles. As shown in FIGS. 1A-1D from four views, 4-hole locking mini plates 1 are shown with spikes on the undersurface for initial fixation and raised borders around locking holes for improved threading of the locking screws and locking guides. The plates have optimal bend to accommodate spinolaminar junction. These embodiments may be Symmetrical XS plates.

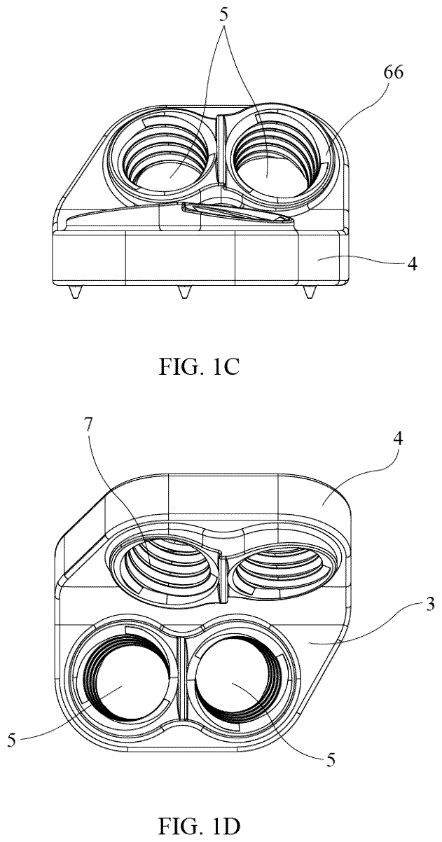

FIGS. 2A-2D depict a bone plate system that includes a bone plate with a screw inserted in each screw hole of the bone plate, in accordance with non-limiting embodiments of the present application, from different angles. These embodiments may be a Symmetrical XS plate with locking screws. Depicted are 4 hole plates with locking screws in place. The screws can be variable in diameter and length. Typical screws may be for example be 3.5-4 mm in diameter and 6-12 mm in length

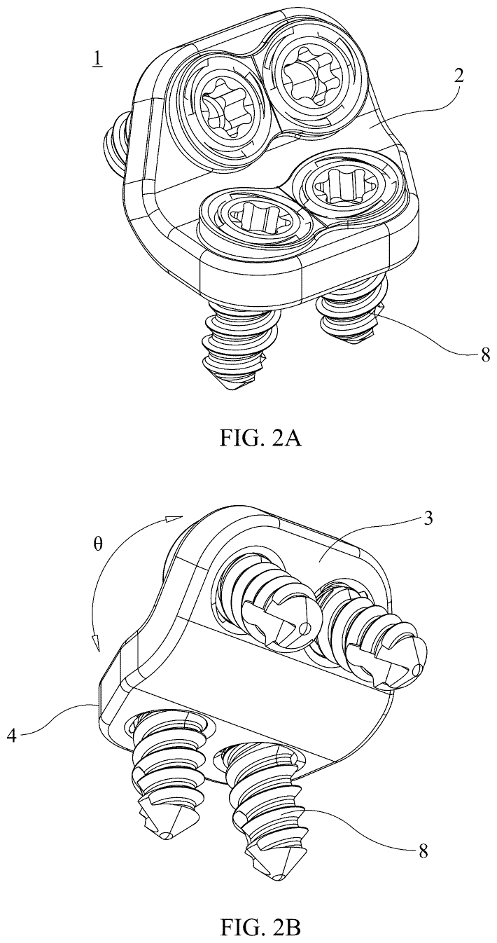

FIGS. 3A-3D depict a bone plate system that includes a bone plate having a guide 21 corresponding to each screw hole of the bone plate, in accordance with non-limiting embodiments of the present application, from different angles.

These embodiments are a symmetrical XS plate with drill guides and locking plates with preloaded drill guides. The guides are removed after using a drill/awl to create a pilot hole.

Example bone plates may include an offshoot section from the main body. The offshoot section may also have one or more offshoot screw openings that have threading configured to accept an offshoot screw(s) or screw-in attachment(s) therein. In non-limiting examples, an offshoot section of the bone plate may be twisted axially with respect to the main body.

FIGS. 13A-13D for example depict an outrigger bone plate including multiple screw holes 5, from various angles in accordance with example embodiments. Example plates have a first side 3, a second side 4 and screw holes 5 therein having threading 7. These embodiments also have an offshoot 9, with at least one screw opening 10 therein having threads 11. The small outrigger bone plate may be a precontoured locking plate that includes 2, 3, 4, 5 or more holes for optimized bony fixation. They are anatomically precountoured to accommodate the posterior spine elements-lamina, spinous process, facet, pars, lateral mass. The following are specifications for the plate of FIGS. 13A-13D.
Minimalistic small plate.
Size is almost perfect.
Adjust spacing between holes to make it symmetric. Large spacing to smaller spacing
Holes should be divergent for increased purchase
Smallest original plate, 5 holes.
Outrigger doesn't fit spinous process
Works well for pedicle screw
Holes should be divergent for increased purchase
Smallest original plate, 5 holes
10, 12 mm screws (color coded)
Keep small original plate only
Smallest original plate, 5 holes, LH and RH versions
Larger outrigger plates are too large
Keep small original plate only

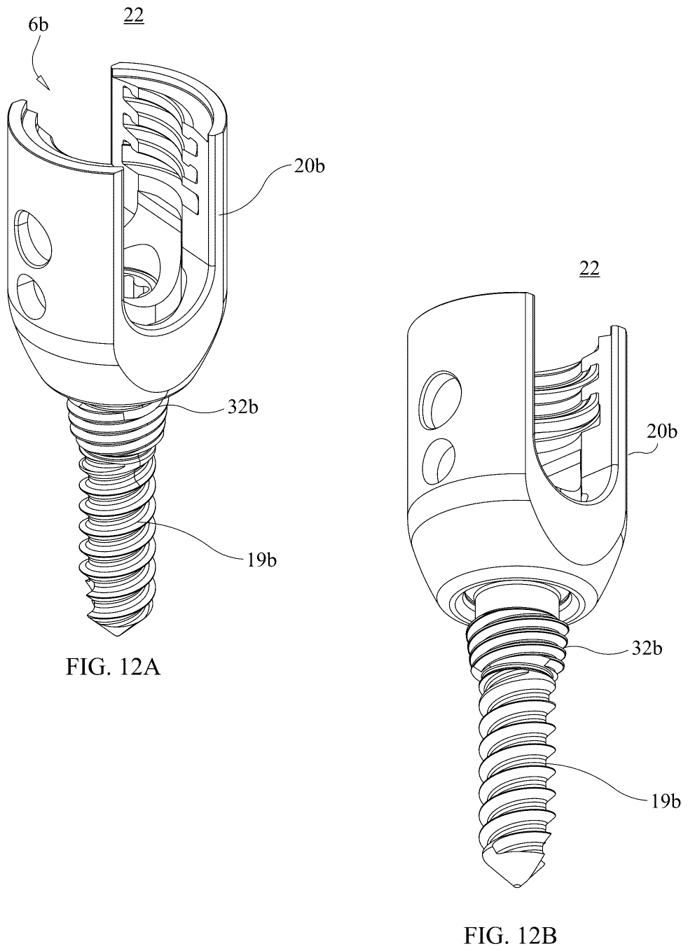

FIGS. 14A-14B depict a larger example of a pre-contoured six hole locking plate with threaded holes 5 in accordance with example embodiments. These embodiments are an example of a pre-contoured six hole locking plate with threaded holes in convergent and divergent configuration as well as undersurface spikes for improved initial stability. Example plates have a first side 3, a second side 4 and screw holes therein. These embodiments also have an offshoot 9, with at least one screw opening 10 therein having threads 11. A first or second side may have one or more additional openings 12. Embodiments include a minimalistic large plate that include.
6, 8, 10, 12 mm screws.
Sizes color coded.
Holes should be divergent for increased purchase

Non-limiting examples of bone plates may further include at least one tulip on a top surface of the main body as an integrated part of the bone plate or a tulip screw having a tulip head on a top surface of the main body. The at least one tulip or tulip screw may be for example rigid or semi rigid and may be of the same or different material as the material of the plate.

Having a tulip associated with the present plates, either with the plate including a tulip or with a tulip screw being included in a screw hole of the plate, enables attachment of for example, a rod, rail or bar to the tulip and therefore to the plate. If more than one plate is used having such a configuration, a rod, rail, bar or other device may connect the plates to each other. A tulip incorporated in a plate or attached thereto may also be used to connect a bone plate to an already existing system. In example embodiments, the tulip serves for rod attachment to connect a plate with other plates or constructs, including for example new or already in place plates or constructs.

According to example embodiments, at least one surface of the main body may be roughened, which may increase bone integration. For example, a bottom surface of the bone plate, which bottom surface is the side of the bone plate adjacent to bone, may be made of a material that is rough, it may be roughened after preparation of the bone plate, or a coating or other addition may be added to the bottom surface of the bone plate. Example locking plates may have rough textured surfaces created by either additive (3D printing) or subtractive (acid etching) manufacturing on all surfaces of the plate which would facilitate plate adherence to the lamina below and bone graft above. The plate may be 3D-printed to provide surface topography features for optimal osteo integration. It may also be CNC-machined, cast or molded. The plate in these embodiments may have both smooth as well as threaded holes. These holes also may be of different diameters. Some of the holes may be used for definitive screw fixation. Other holes may be used for threading a plate inserter or drill guides.

According to example embodiments, at least one surface of the main body may be smooth.

According to further embodiments, the bone plate may include a at least one spike or a plurality of spikes that may assist in initial fixation of the bone plate to bone of the patient.

Plates in accordance with the present application may include other surface protruding features and/or surface roughness/texture. In example embodiments, the undersurface of the plate may be smooth or textured with surface topography features created by additive or subtractive manufacturing to create rough surface for osteointegration. It may also have one, two or plurality of spikes/protruding elements for initial stability. Plates having possible spikes and/or surface roughness or texture may apply to various embodiments herein.

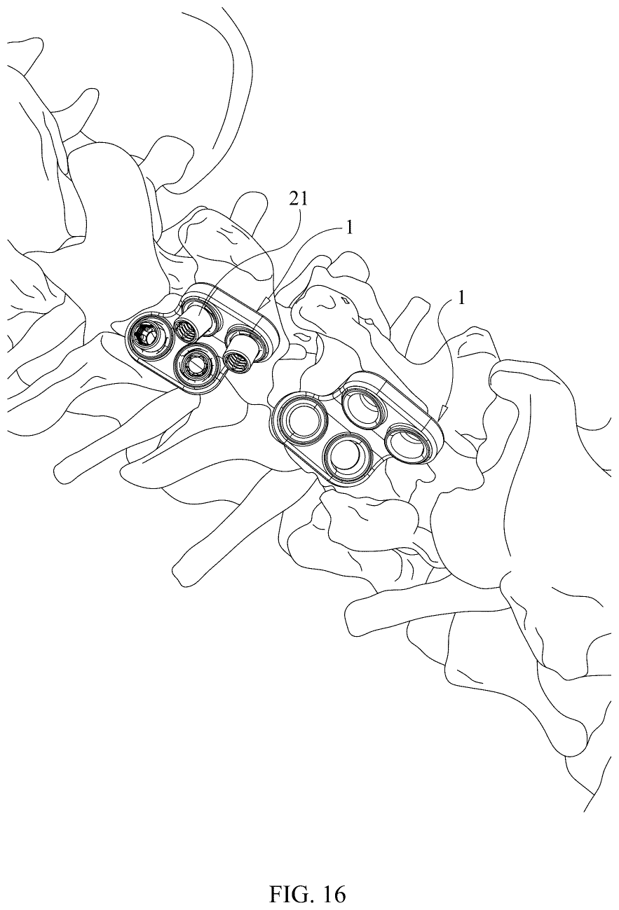

FIG. 22 depicts a blocking plate 38 with a tulip 41 (poly or monoaxial) on top of the blocking plate, in accordance with example embodiments. This embodiment illustrates the use of a locking cover plate to minimize screw backout, especially if non-locking lag screws are utilized, rather than locking screws. The blocking plate may be attached to the top of a bone plate 25 in example embodiments. Also shown is a screw in eyelet 34 having a screw portion 35 for cable, suture or band passage through an opening 36. The screw-in eyelet may be screwed into a screw hole 27 of a main body 26 of a bone plate 25, which allows for cable, suture or band passage. A bottom portion of the blocking plate 40 (which may be the bottom of a tulip screw passing through the blocking plate 38) may be configured in size and shape to engage with a corresponding opening 42 in the bone plate 25.

Further shown are spikes 29 for initial fixation of the bottom of the plate on the bony surface. FIG. 22 depicts the use of a blocking cover plate 39 to minimize screw backout, especially if non-locking lag screws are utilized.

According to example embodiments, the main body may further includes a hook or hook plate, which may be configured to engage spinal lamina of the patient. The hook or hook plate may be the same or different material as the bone plate. The hook or hook plate may also be integrated with the bone plate, or it may be an attachment thereto (for example it may be part of a kit and selected and attached to the bone plate prior to insertion in the patient).

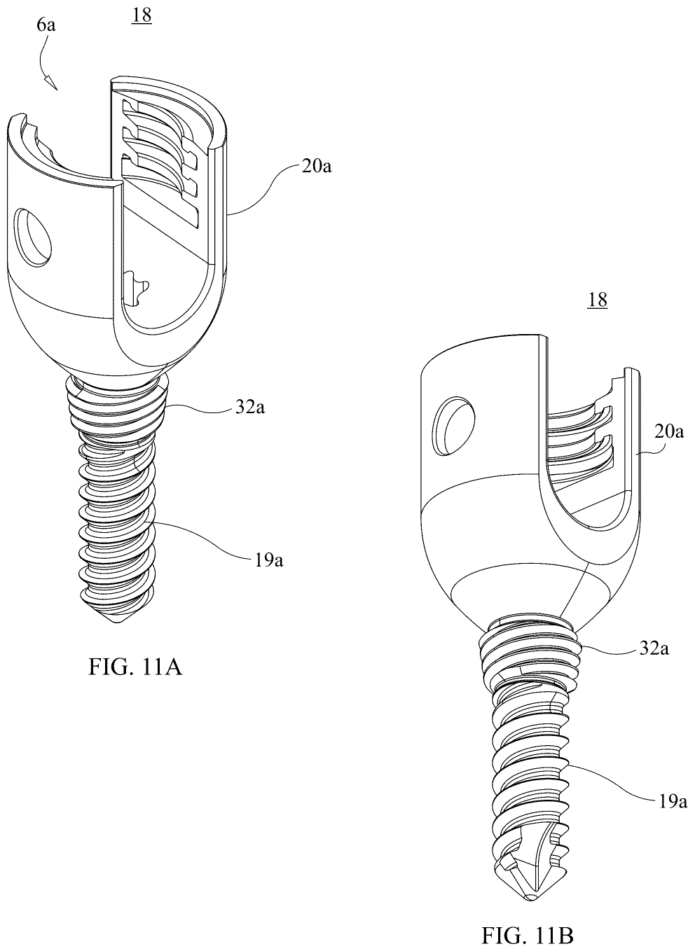

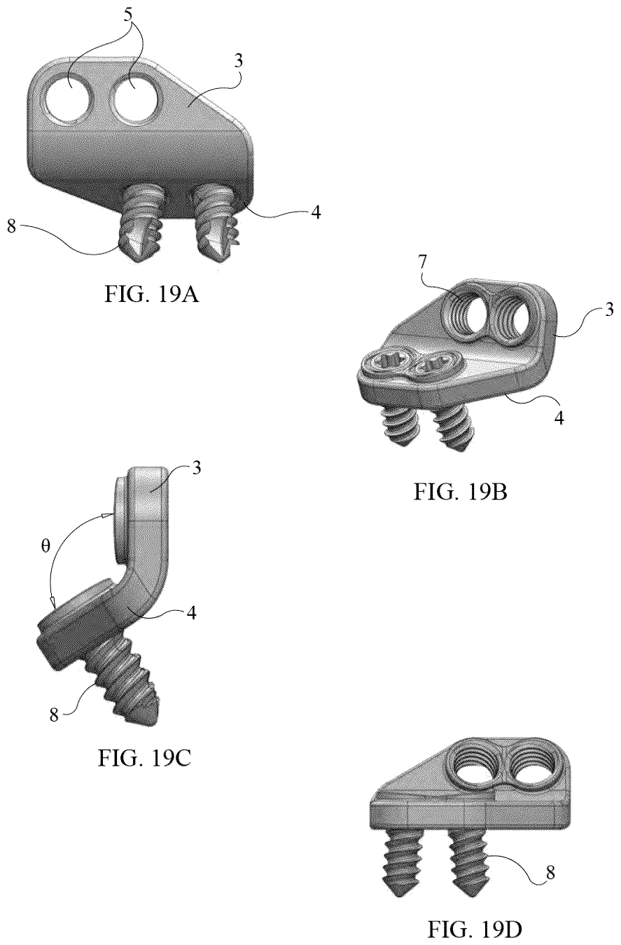

FIG. 25 depicts a mini hook plate in accordance with example embodiments. In particular, FIG. 25 depicts a spine plate 25 having a tulip screw having a tulip 43 engaged therewith, having one or more screw holes 27, and a curved hook 33 on the end of the bone plate to engage spinal lamina. FIG. 12 depicts a gradient of stiffness formed in a patient. A spinolaminar plate can accommodate one, two, three or more tulips that can accept rods/rails of different material and diameter, Therefore, one can use stiffer and stronger rods at the foundation of a long construct.

For the levels with lesser bone density at the upper end of a long construct in cases of Adult Spinal Deformity (ASD), a gradient of stiffness may be created according to non-limiting example embodiments. A spinolaminar plate can accommodate for example, one, two, three or more tulips that can accept rods/rails of different material and diameter, Therefore, one can use stiffer and stronger rods at the foundation of a long construct, possibly running triple or quadruple rods on the bottom (ie T10-pelvis). The upper softer part of the spine will be instrumented with dual titanium rods to create a gradient of stiffness to mimic closer native spine rigidity/stiffness and minimize proximal junctional failure. This construct of variable stiffness is made possible by having two or more polyaxial or monoaxial tulips per side per level, which allows for combining of different number of rods and different rod materials at different levels.

According to example embodiments, the bone of a patient to which the present bone plates are attached may be spinal bone, such as spinolaminar applications of the present plates. For example, the present bone plates may be used as part of a plate locking screw system for posterior spinal fixation. Non-limiting embodiments of the present application include patient-specific spinolaminar locking plates, methods and systems for segmental fixation of the spine. The terms “spinolaminar” and “spinal laminar” are used interchangeably herein.

According to other embodiments, the bone of a patient to which the present bone plates are attached may be non-spinal bone. For example, the bone(s) may be extremity bone(s), pelvic bone(s), or rib bone(s).

Example bone or locking plates provided herein may be of various shapes and sizes.

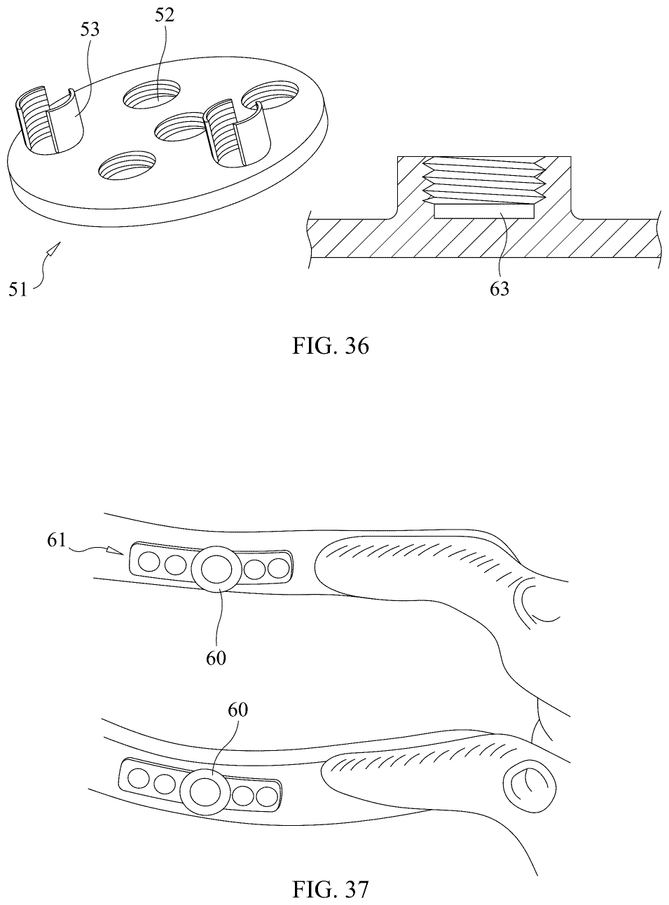

FIG. 37 for example depicts a linear locking plate 61 according to non-limiting example embodiments. The term linear means that the locking plate is generally or essentially linear and need not be exactly linear. The locking plate may also include a rib anchor for VEPTR/Derotation. Also included in the plate system of FIG. 37 may be a polyaxial/monoaxial tulip 60. These embodiments may be used for example in scoliosis patients, who may be too young for spine fixation. Linear plates may be used for rib fixation for scoliosis/kyphosis correction and spine derotation/reduction maneuvers.

A similar construct can be used for spine derotation to correct an axial deformity such as seen in adolescent idiopathic scoliosis, in accordance with example embodiments. In this application the spinal laminar plate can actually be attached at the ribs or costovertebral junction and engage both the ribs as well as transverse processes and lamina. Additionally, a locking costal vertebral plate can be used for similar application such as VEPTR—a vertical expandable titanium prosthetic rib—used for early onset scoliosis or kyphosis, congenital deformities and thoracic insufficiency syndrome.

In the present systems kits and methods, devices or elements are the same as otherwise described herein.

### Bone Plate Systems

Also provided are bone plate systems that include a bone plate as described herein and at least one screw or screw-in attachment.

Screws that may be used in accordance with the present application may include for example locking or lag screws or combinations thereof.

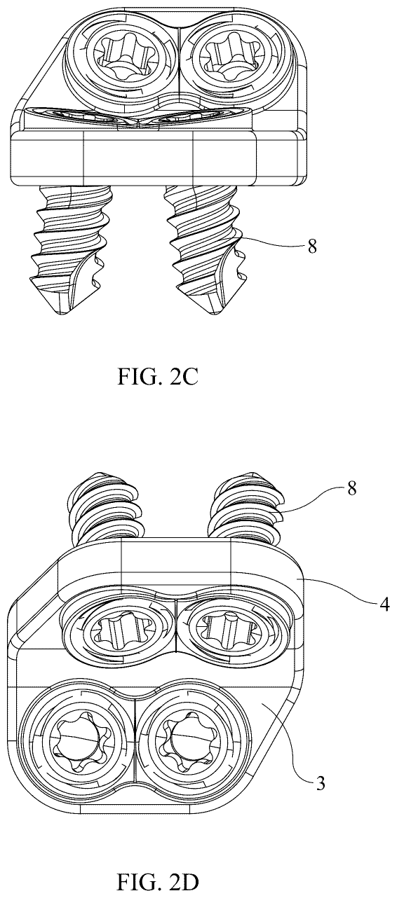

FIGS. 4A-4D depict example locking screws 14 having a lower screw portion 15 and threaded heads 16 to engage threads on a plate, in accordance with non-limiting embodiments. The screw tip can be blunt or sharp. The screws can be fully or partially threaded or completely unthreaded (locking pegs). The screws can be regular, self-tapping or self-drilling.

The locking screws 14 also include a top portion 13, configured to correspond to a tool or screw driver to allow one to screw in or out the screw.

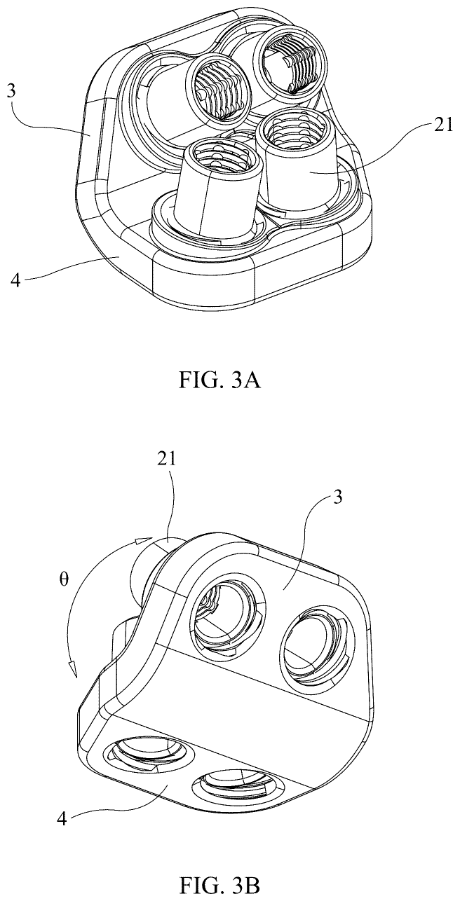

FIGS. 5A-5D depict example non-locking lag screws 17 that may be used with the present bone plates, in accordance with non-limiting embodiments. Screws without the thread in the screwhead are referred to herein as “lag screws”. Non-locking screws can be used with plates as well. FIGS. 5A-5D show lag screws 17 that will help bring the plate closer to the bone, before placing the locking screws. this is an example of a lag screw in locking hole that is used to cinch down the plate. A lag screw may include lower threading 15 and have an upper non-threaded portion 24.

The non-locking or lag screws 14 also include a top portion 13, configured to correspond to a tool or screw driver to allow one to screw in or out the screw.

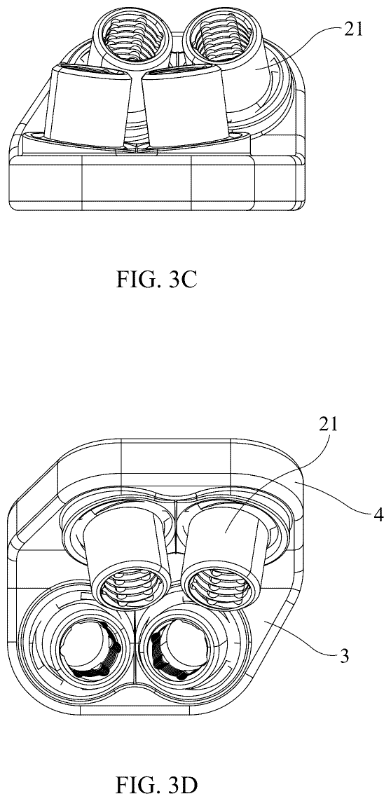

FIG. 6 depicts an example of a lag screw in an example bone plate system of the present application. This shows a spine plate 1, with a preloaded drill guide 21 and a tulip screw 18. A non-locking lag screw engaging the threaded hole in the plate is shown. Also undersurface spikes for initial engagement of the bone are shown. The lag screw may be sized to interfere with plate threads as a screw head comes even with the plate base. FIG. 6 also depicts a cross section of a lag screw 23 for possible use with the present systems and methods in example embodiments. The lag screw has no threads in the head of the screw and is not threaded into the cross-section of the plate threads. The screw lags/cinches the plate down to the bone. In contrast, if one were to lock into the plate, then it wouldn't push the plate down, it would stop.

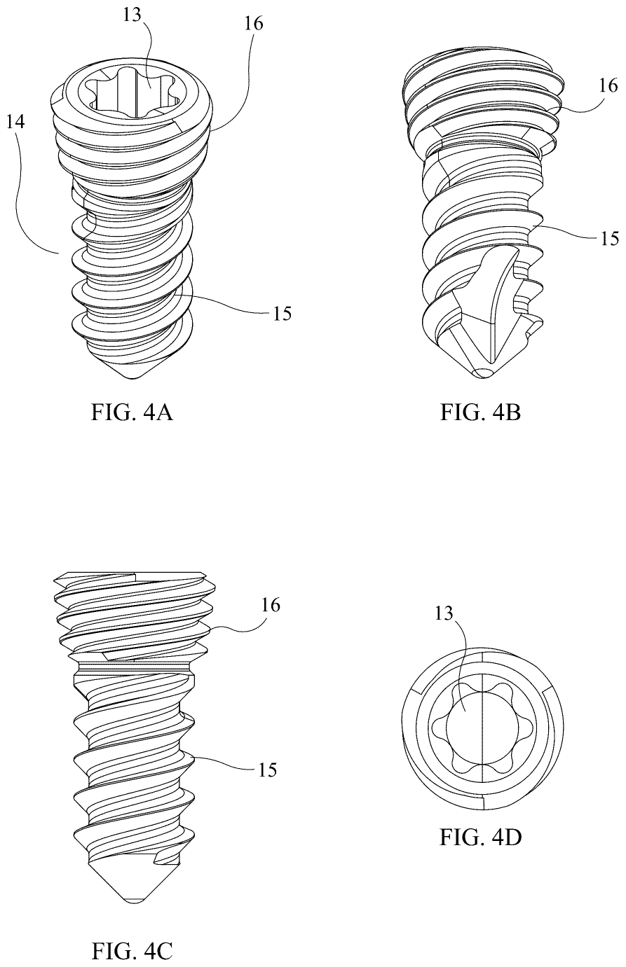

FIG. 7 depicts example screws that may be used in accordance with the present application. Depicted are a combination of locking and non-locking screws, some fully threaded and some partially threaded. FIG. 7 includes locking, non-locking, fully threaded, partially threaded and tulip screws (tulips removed). A close-up picture shows a fully threaded self-tapping locking screw. Also contemplated are locking pegs-similar to locking screws in terms of threads in the screw head, but no threads on the shaft.

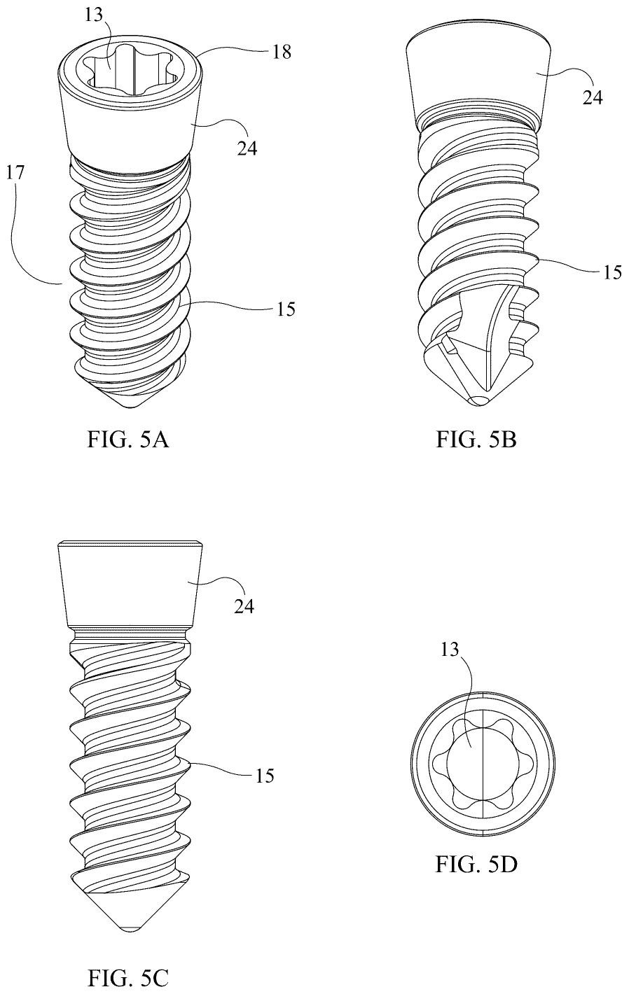

FIG. 8 depicts example screws that may be used in accordance with the present application, including optional sizes, such as diameter sizes, of the screws. The sizes of the screws can also be smaller than 3.0 mm in diameter (especially for the cervical applications) and larger than 4.0 mm in diameter, but examples with diameters within that range are shown. The screw may be a dual lead, still 0.5 mm thread depth, with effective pitch up form 1.5 to 2.5. 6-20 mm lengths.

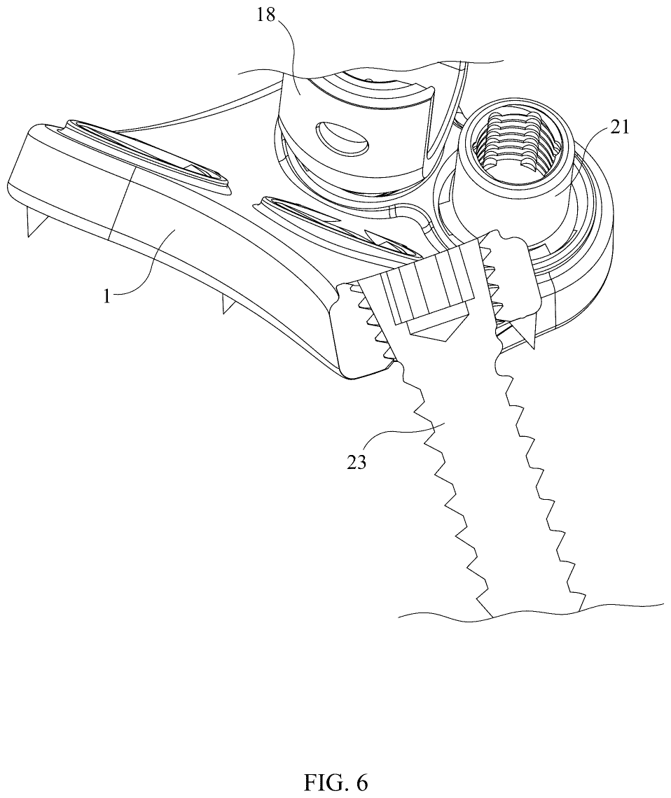

FIG. 9 depicts a combination of locking and non-locking screws that may be used in accordance with the present application.

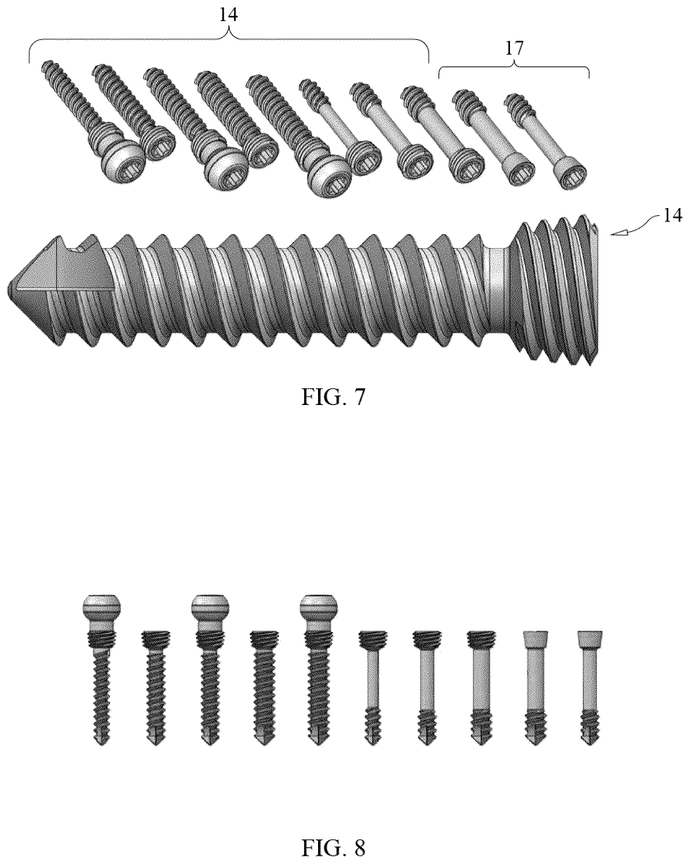

FIG. 10 depicts both locking and non-locking screws of various diameters in accordance with the present application. Smaller diameter screws can be used for cervical spine and larger diameter screws can be used for extremity fixation. On the left, threads may taper out at 20 deg. At about 1 mm from the head of the screw, to maintain maximum shaft diameter. Bone threads are all dual lead, 2.5 mm pitch. The left two screws may have for example. 3.0 mm, 2.4 minor diameter, 0.3 mm thd depth. The next two screws from the left may be 3.5 mm, 2.8 mm minor diameter, 0.35 mm thd depth. The right two screws are 4.0 mm, 3.0 mm minor diameter, 0.5 mm thd depth. The right most screw has a small undercut required for 4.0 mm due to taper thread size.

3.5 and 4.0 screws may be used for example, for fixation and locking into the plate, cold welding and creating a very stable implant that has high pull out strength.

Other example screws that may be used in accordance with the present application may include for example tulip screws, such as monoaxial or polyaxial tulip screws.

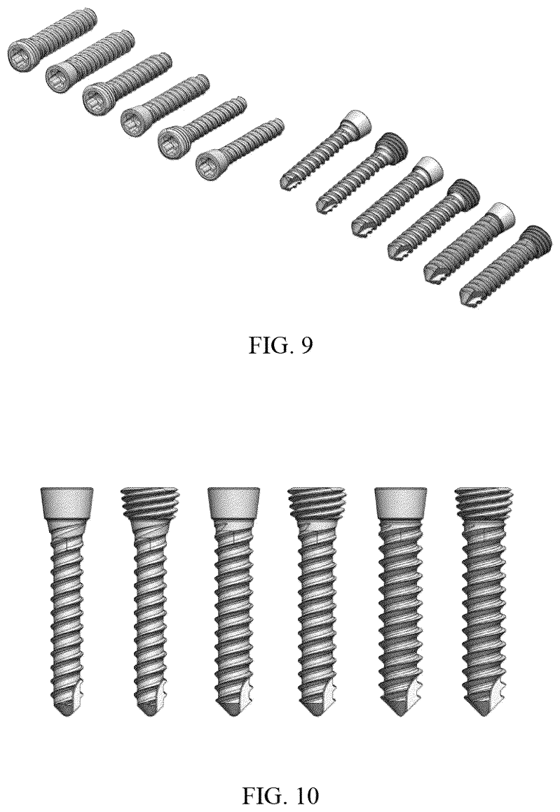

FIGS. 11A-11D depict monoaxial tulip screws 18 from various views that may be used in the present systems according to example embodiments of the present application. Monoaxial tulip screws incorporate a tulip 20a for rod/bar engagement, a threaded head 32a for plate engagement and threaded shaft 19a for bone engagement. The threaded head 32a that is configured to engage with the plate, is unique to the present tulip screws. Custom plates for use with monoaxial tulip screws may be developed e.g., using CT and densitometry. The tulip 20a defines an opening 6a in which a rod, bar or other element may be engaged.

FIGS. 12A-12D depict polyaxial tulip screws 22 that may be used in the present systems according to example embodiments of the present application. Polyaxial tulip screws also incorporate a tulip 20b that define an opening 6b for rod/bar engagement, a threaded head 32b for plate engagement and threaded shaft 19b for bone engagement. The threaded head 32b that is configured to engage with the plate, is unique to the present tulip screws.

Polyaxial tulip screws may have a rotatable tulip head with respect to the threaded head of the screw, to position the tulip head to a desired position. The rotatable tulip head may be rotatable with respect to the threaded head via a ball and socket or other connection. The rotatable tulip head may be locked to a desired position for engagement of both the threaded head 32b with the plate and a rod or other attachment with the tulip head, so the rod or other attachment may be fixed by the polyaxial tulip screw at a desired position with respect to the bone plate to which the threaded head is attached.

Example embodiments may include a locking tulip button. In these examples, the tulip screw may include a tulip in which the tulip screw has a threaded head but no shaft. In such example embodiments, tulip screws may have a tulip for rod engagement, a threaded head for plate engagement, but no shaft for bone engagement. Such example tulip screws may be useful for example for cases in which a patient has compromised bone, such as in a patient who had a prior laminectomy, in which there may not be a sufficient landing zone for a regular tulip screw having only threading for engaging bone.

The screw or screw in attachment may each correspond to a respective screw opening of the plurality of screw openings in the bone plate. The screw(s) and/or screw-in attachment(s) may be selected for example based on the surgery being performed that the goals to be achieved.

In example embodiments, a screw-in attachment may include a locking eyelet for example having threads for screwing the locking eyelet into a screw opening of the plurality of screw openings, and having an eyelet opening configured in size and shape to allow a band, wire or cable to pass therethrough.

Another example of a screw-in attachment may include a blocking plate having for example, a protrusion or screw on a bottom side of the blocking plate and an attachment mechanism on a top side of the blocking plate. The protrusion or screw may be attachable to an opening defined on a top side of the bone plate so that the bottom of the blocking plate engages with the top of the bone plate. In example embodiments the attachment mechanism at the top of the blocking plate may be a tulip, but other forms of attachment are contemplated.

In examples of the present systems, each screw opening has a corresponding screw or screw-in attachment. In example systems, the screws or screw-in attachments may all be the same or they may be different. For example, there may be several of one type of screw, and another screw and a tulip screw or eyelet all screwed into the same plate within a system.

In example embodiments a bone plate system or locking plate system can have a tulip screw placed in any of the screw holes which provides variability and functionality benefits. This makes it easier for the tulip to attach to other rod constructs or to standalone in example embodiments. The bone plate can be positioned to optimize rod attachments, and in embodiments the tulip can be placed in any of the holes in the plate, this will help align attachments e.g. via cross-connector.

In example embodiments, the present systems may provide optional or variable positioning of tulip hole placement, which will ease connection to rod constructs when used as a supplement to improve overall fixation. Example embodiments also provide optional/variability in where the tulip can be placed, which will make rod-to-rod connections easier to align.

The tulip in example embodiments may have a flat blunt surface that locks into the plate and/or screw that protrudes beyond the plate. Either option can be used based on the situation encountered by the surgeon. The locking screw tulip will provide additional fixation just like a standard screw passing through the plate. The blunt tulip option can be used in areas where there is no fixation if the plate were to lie in such an area but still allows the tulip to lock into the plate without a screw if necessary.

The locking tulips provided herein may serve as a point of attachment to segmental plates and can be monoaxial or polyaxial. In example embodiments, more than one tulip can be placed onto a single segmental plate—a unique feature not available with conventional screws or hooks.

Examples of systems in accordance with the present application may include one or more drill guides corresponding to at least one of the screw openings. Example bone plate systems may include a bone plate pre-loaded with at least one drill guide, prior to insertion of the plate to a patient, to assist in application of the bone plate to bone of a patient. Each drill guide may correspond to a screw opening and may be for example smaller, bigger or the same size as a corresponding screw opening. The term drill guide should not be limited to guiding “drills” but may also help in guiding one or more other tools to a screw opening, such as an awl. In example embodiments, there may be multiple drill guides corresponding to several or even all of the screw holes in a bone plate. Drill guides may also be attached to the plate at an angle that corresponds to the angle of the screw holes. Accordingly, the drill guides may be at oblique angles with respect to one another and/or with respect to a surface of the main body. According to example embodiments, the drill guide(s) are detachably attached to the bone plate, such that they may be removed from the bone plate after use.

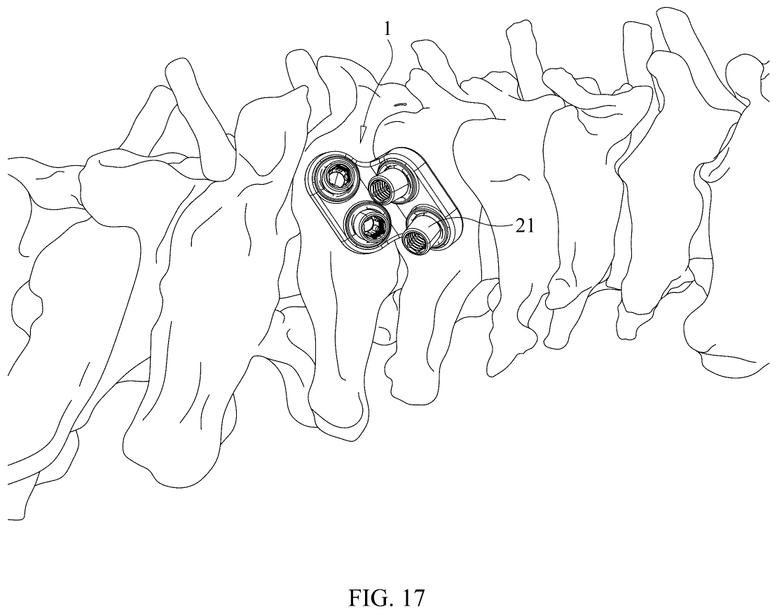

FIG. 23 depicts a plate according to non-limiting example embodiments that has pre-loaded drill guides 30 screwed into a plate 25, which may streamline implantation of the plate.

As can be seen in FIG. 23, example plates may also have spikes 29 on an opposite side of the bone plate from the pre-loaded drill guides, for initial stability. The undersurface of the plate in this example, may have 1, 2, 3 or more spikes/protruding surface features for initial stability.

In example embodiments, driver guides or drill guides may be designed to not fight each other when implanting the screws in multiple holes at the same time.

Example systems may further include a second or more bone plate that may also have at least one screw hole and at least one screw or screw-in attachment. Systems having multiple bone plates may also have for example, one or more rods, bars or rails that may connect to the multiple bone plates.

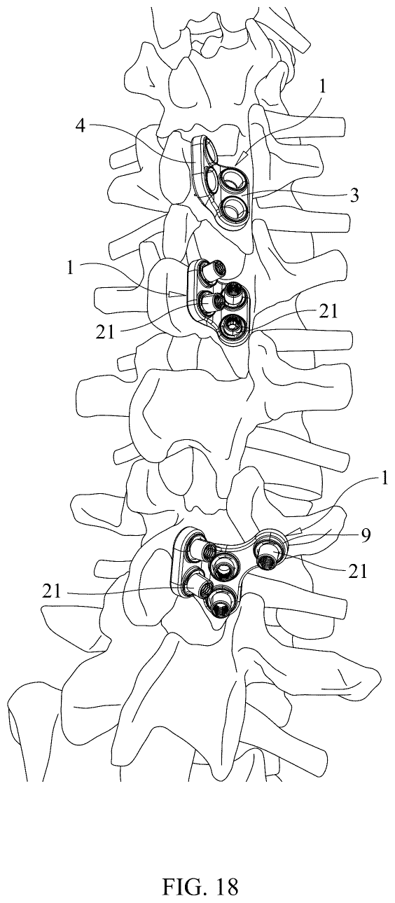

FIG. 24 depicts a system in which spine plates can be combined with other segmental anchors such as pedicle screws in the same construct. Also it shows that a pedicle screw can be used with the plate at the same level and same side. FIG. 24 depicts a hybrid construct using example bone plates along with a pedicle screw used at the same level and same side as the bone plate with plate. The hybrid construct is with a pedicle screw used at the same level and same side as the plate with plate and screw connected via a lateralizing or iliac-type offset connector.

FIGS. 15-18 depict examples of 3-D printed plate on a spine model in accordance with examples of the placement of different spinal bone plates, having different configurations and some with guides and some without guides, at adjacent segments of the spine. These figures show an example of spinal plate placement at the adjacent segments of the spine. Note that plates serve as segmental anchors (covering only one spinal segment each) instead of being bridge plates spanning across several segments. An exception to spanning one segment may be in examples in the cervical spine, where the plating system can be used standalone to span two lateral mass segments, as the plate is large enough to do that.

In FIG. 15, the plate on the left shows preloaded drill guides in accordance with example embodiments. FIG. 16 depicts bent offset connectors, one with drill guides (on the left) and one without (on the right).

FIG. 17 shows a bent offset connector and demonstrates that a plate can be also used as a bridge spanning two segments together, especially in the cervical spine.

FIG. 18 depicts a bent offset connector and outrigger. FIG. 18 illustrates combining plates of different sizes with different number of holes in the same construct

FIG. 25 depicts a gradient of stiffness formed in a patient. A spinolaminar plate according to example embodiments, can accommodate one, two, three or more tulips that can accept rods of different material and diameter. Therefore, one can use stiffer and stronger rods at the foundation of a long construct.

The present systems relating to the connection of the attachment component variability (plate to other system or a second plate is unique. Because the attachment can be placed in multiple holes, depending on what you're trying to accomplish: a standalone device and/or attaching as a supplementation to support another system (or both with two tulips screwed into plate). The screw in part of the attachment that allows one to connect the system to itself or a supplemental system may vary,

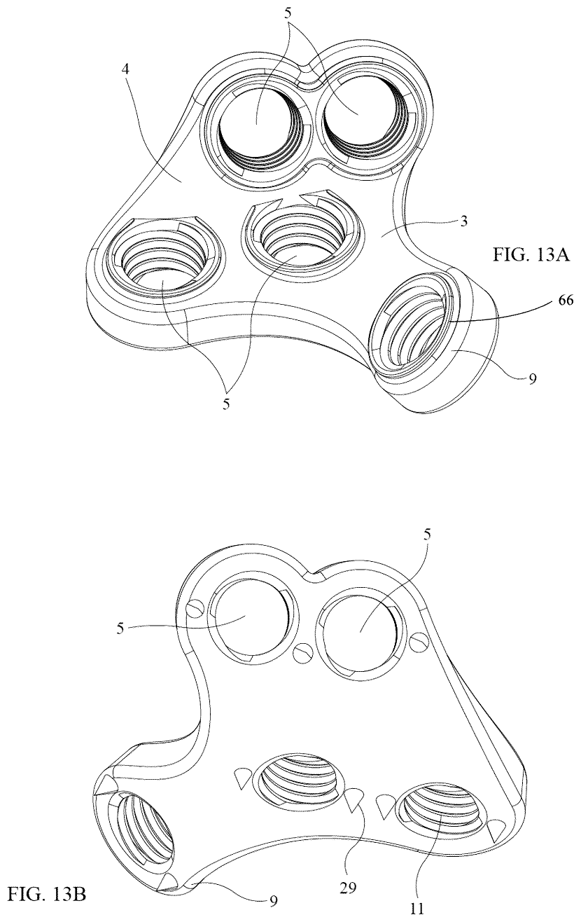

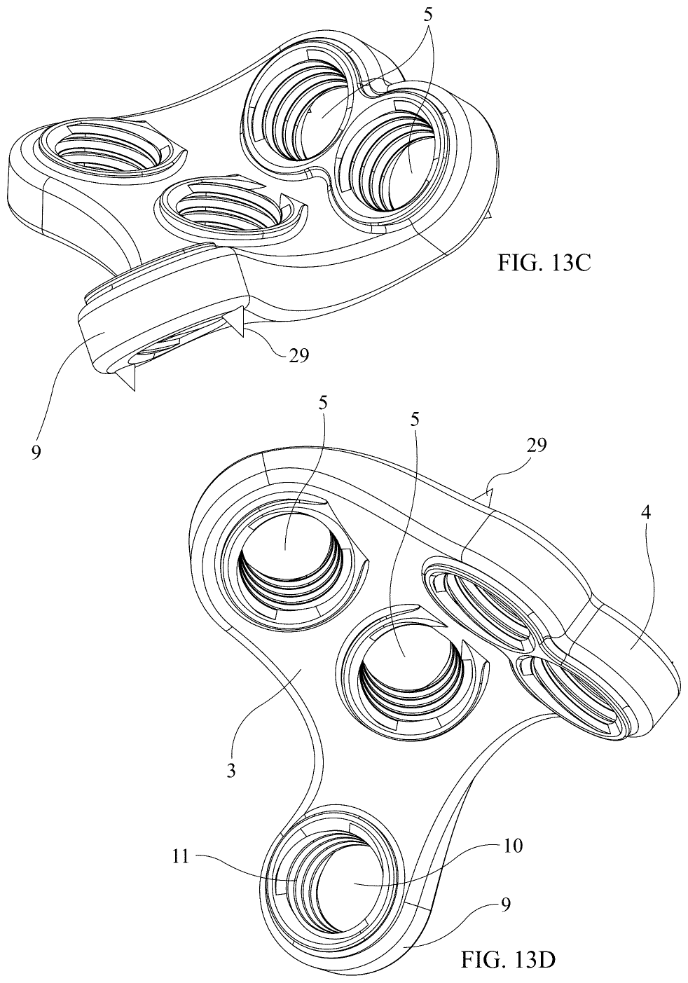

FIGS. 19A-19D depict an example of a 4-hole asymmetrical bent bone plate from four views, with divergent screws in holes of the bone plate, in different planes. The divergent screws in these embodiments in different planes improve pullout. These embodiments include a raised border around the threads to add depth to the threads without making plate thicker.

FIGS. 19E-19I depict a see-through rendering of an example of a bent offset 4-hole bone plate from five views, with divergent screws in holes of the bone plate. FIGS. 19E-19I show contours which may have different angles of for example, 100-125 degrees.

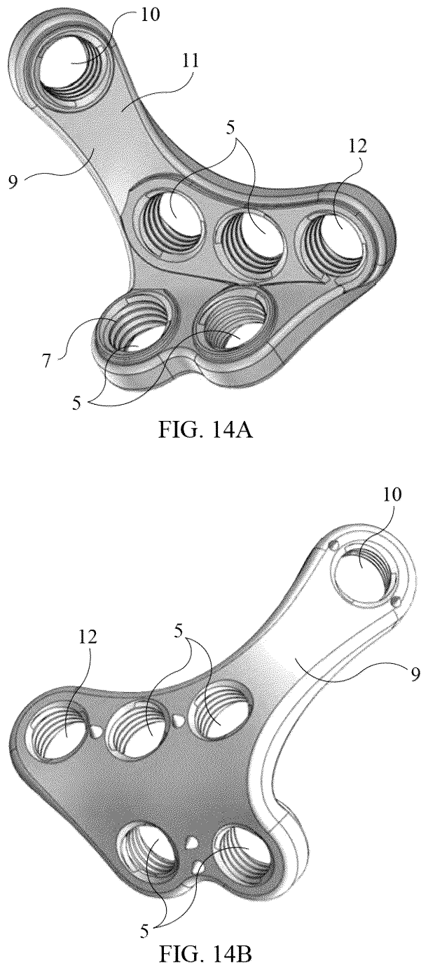

FIG. 20 depicts a plate having screw holes and an eyelet screw-in accessory that may be screwed into a screw hole, for a sublaminar band, wire, suture or cable passage. FIG. 20 demonstrates the use of bands 37 attaching to the plates 25 via threaded locking caps 34. This figure shows a small threaded knob 26 instead of a screw and passing a braided tape/band 37.

FIG. 20 depicts a plate 25 having screw holes 27 (the shape and configuration of which may be varied as described herein) in the main body 26 of the bone plate and an eyelet 34 for sublaminar band, wire, or cable passage 37. The holes 27 have threading 28. The eyelet may be of various shapes so long as it is configured to accommodate a sublaminar band, wire or cable, and may attach to the plate, for example by screwing into the plate. The bottom of FIG. 7 also shows a side and front few of a non-limiting example of an eyelet.

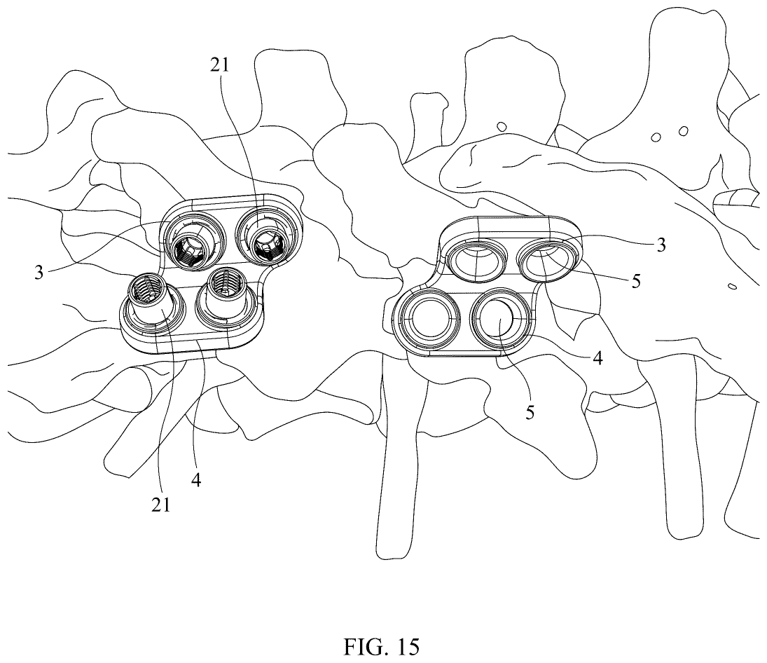

FIG. 21 depicts a plate with another example embodiment of an eyelet with locking threads, which allows for both locking into the plate as well as passage of the sublaminar band. Locking threads can be engaged in any of the locking holes in the spinal laminar plate.

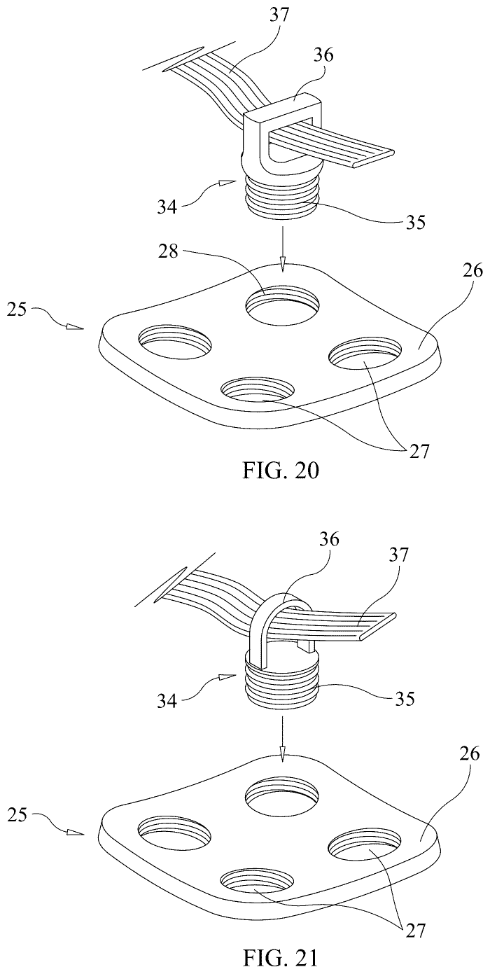

FIG. 27 relates to tethering applications in which a locking plate 25 can also be applied to the vertebral body or posterior elements either anteriorly, laterally or anterolaterally. The tulip 43 in this application may accept a flexible non-rigid tether such as a braided polymer to allow for motion preservation. This shows anterior use of spine plates. Here the screws are placed into the vertebral body rather than posterior elements. The plate tulips can accommodate rod, bands, cords or rails.

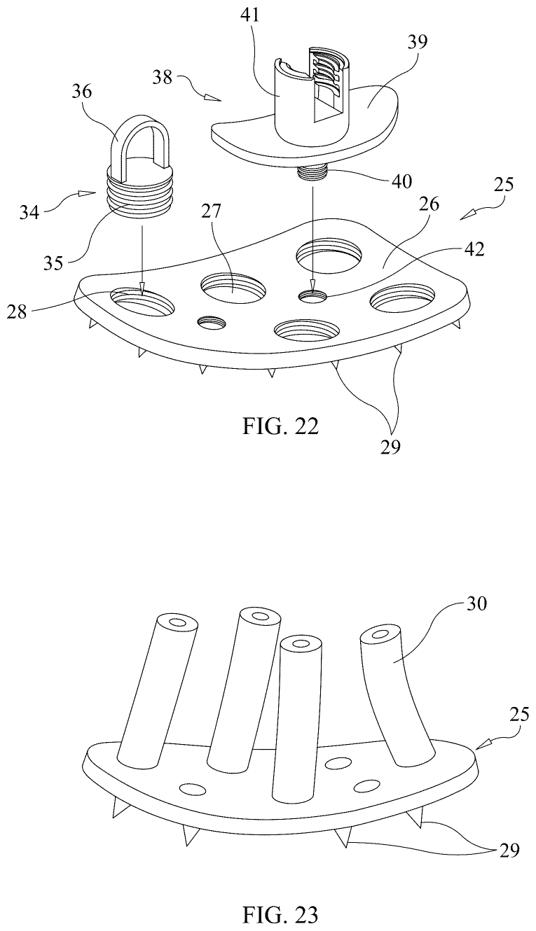

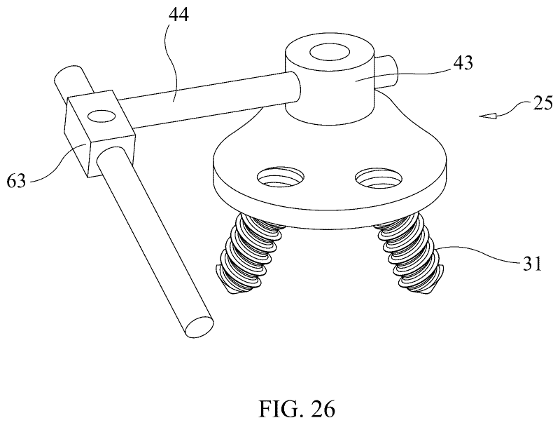

FIG. 28 depicts another example of a hybrid construct incorporating plates and pedicle screws in the same construct. FIG. 30 depicts a mini (or mini-plate) version of example bone plates in accordance with example embodiments, which may be used after laminectomy/Transforaminal Lumbar Interbody Fusio (TLIF) cage placement. Example embodiments may have a limited number of holes and smaller footprint than other example plates. A hybrid construct may have spine plates topping off a pedicle screw construct. Accordingly, the present bone plates can be combined with pedicle screws.

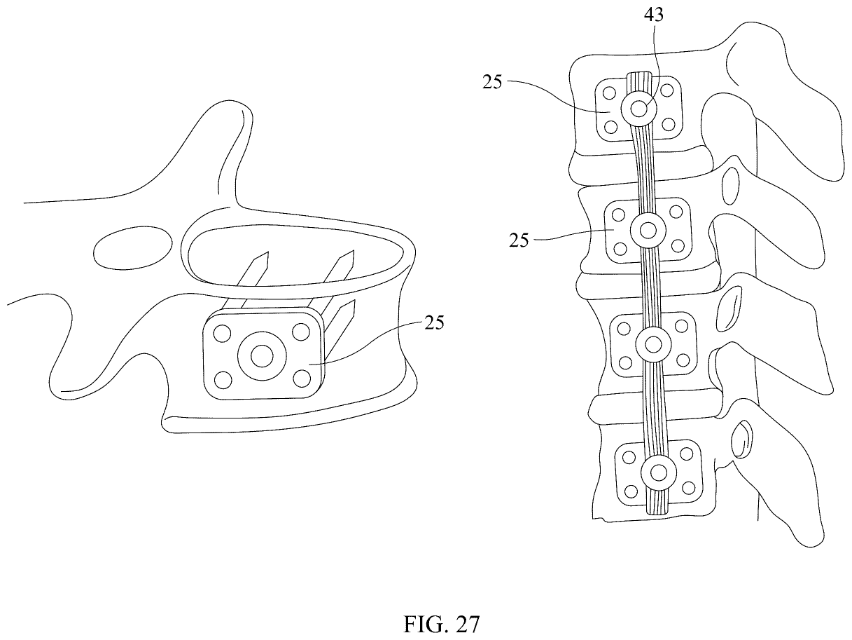

FIG. 31 depicts an example locking screw 46 having a bottom threaded portion 47 and a top threaded portion 48 with a locking cap on top 50 of an opening 49 in the top of the screw that can be used with present bone plates that can have a cannulation to allow for passage of suture 51. As with other embodiments, the example plate may have one or more spikes 29 on a bottom side of the plate

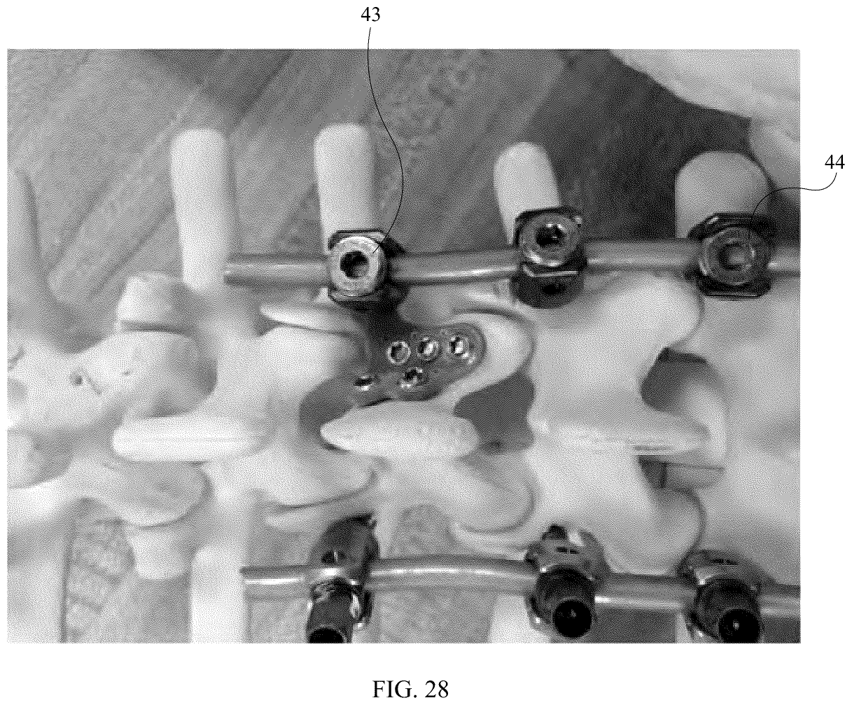

FIG. 32 depicts an example bone plate 51 that may have one or more screw openings and one or more fixed tulips 53 on its surface to provide rigidity. In these embodiments, the tulips are an integrated part of the plate, rather than being part of a monoaxial or polyaxial screw. The rigidity may be desirable for example, in cases of scoliosis correction or fracture reduction. Any given plate may have one, two or more built-in tulips better either fixed angle or polyaxial. These tulips may also be uniplanar or preferred angle. According to example embodiments, the tulips may be made as part of the plate or they may be formed separately from the plate and thereafter affixed thereto. The tulips may be at a fixed angle or may be toggling in different planes/directions according to different embodiments.

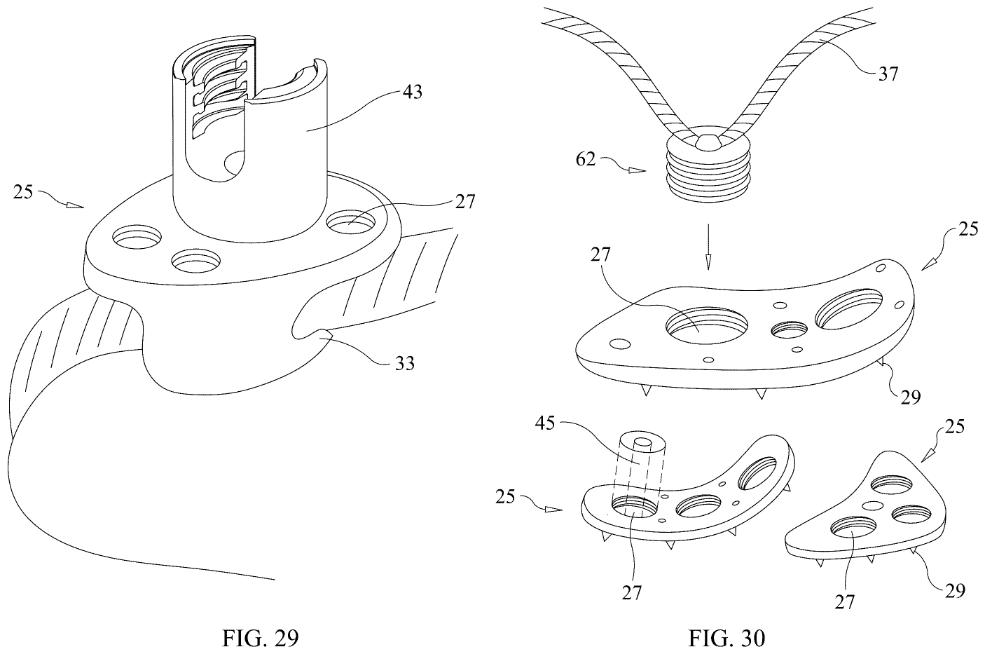

FIG. 33 depicts example embodiments having a monoaxial locking screw 54. The screw may have a fixed angle or an angle tulip that may be configured for receiving a rod or bar or rail therein. The screw may have a threaded portion 55 to engage with bone, a threaded portion 56 to engage the locking hole in the plate. The screw may have a diameter of for example 2.5 to 4.0 mm. Screws according to non-limiting example embodiments will also have a fully-threaded, partially-threaded or smooth shaft 57 extending past the plate to engage the bone. Non-limiting materials may include for example, cobalt, chrome or titanium.

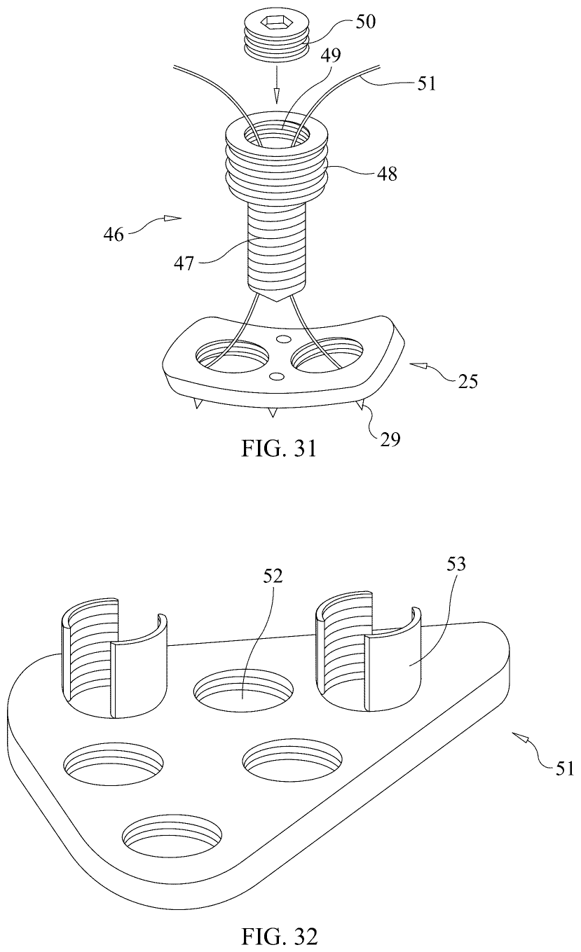

FIG. 34 depicts example bone plates applied for a non-spine application to a long bone, the bone plates having attachments to engage with multiple rods, bars or rails. This example system may be used for example to span a fracture in the long bone. Embodiments include extremity fixation (appendicular skeleton and long bone fixation devices and systems including modular plates, rods and rails. A rod may be used instead of a rod to decrease the prominence and increase stiffness of the construct. systems and methods include examples of using plates for fixation. one, two, three or more rods or rails can be used for the fixation.

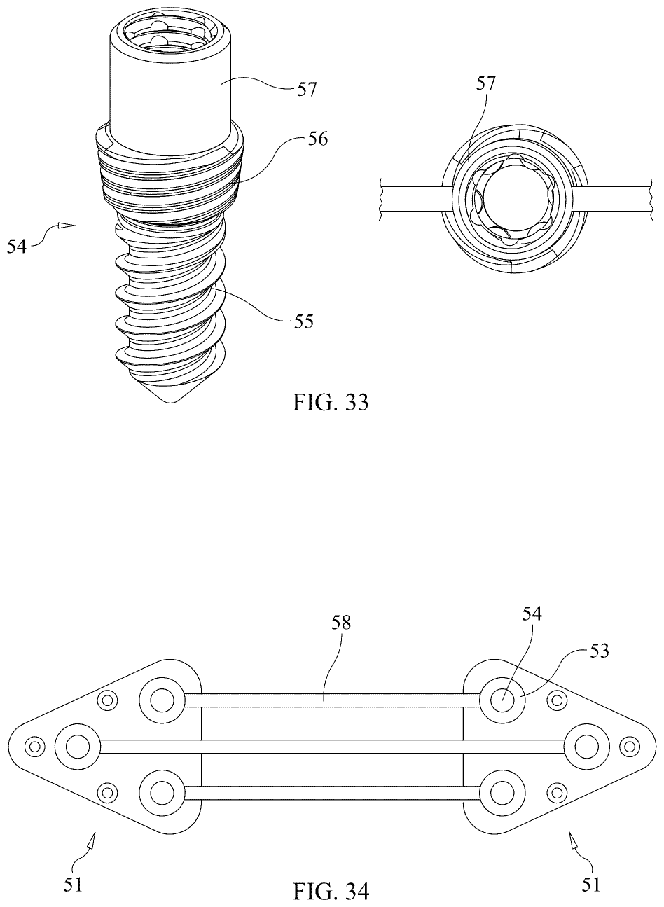

FIGS. 34 and 35 depict example embodiments in which the locking plate can be adapted for cervical spine. It can incorporate a lateral mass screw through a nonthreaded or threaded hole. The lateral mass screw may engage the plate and be a locking type screw. Alternatively can be a conventional nonlocking screw placed through the plate.

FIG. 34 depicts a non-limiting example of locking plates 51 applied to e.g., a long bone with three rods or rails 58 spanning a fracture. It should be understood that the plates may be of a different size and/or shape and may include a different number of rods. There may be for example one, two, three, four or more rods/rails within the scope of the present application. The rods in these examples may be as stiff and/or as strong as desired depending on the particular use.

FIG. 35 depicts an example bone plate 51 having one or more fixed tulips 53, and one or more screw holes 52 that may be used for long bone fracture fixed with dual construct of modular locking plates with one, two, three or more rods, bars or rails spanning the fracture.

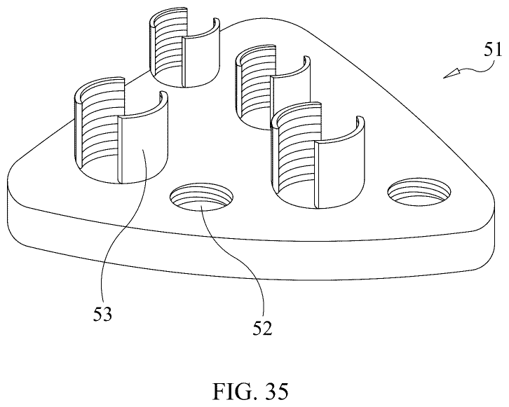

FIG. 36 depicts an example modular locking plate 51 for extremity fracture fixation. A locking set screw can lock a circular, elliptical, rectangular, square or differently shaped bar or rod or rail 63 depending on surgeon's preference and the rigidity of the fracture fixation desired. Such plate can be placed through a small incision and the rod, bar or rail can be passed under the muscle or fascia to minimize soft tissue trauma.

According to example embodiments a rectangular bar may be stronger than other shapes, which may be desirable. A rail may be stiffer and/or stronger than a rod and may also have a smaller profile, and therefore may be desired. According to example embodiments, the bar may be of a trapezoidal or other shape for strength purposes. The rod/rail may be selected based on the patient and based on the desired profile. It is also considered that the size of the rod or rail (profile height and length) may be provided in various size increments, for different patients and uses, such as e.g. in 5-10 mm increments. Additionally, it is contemplated that rods or rails may be tapered toward one or more ends, which may be useful, for example with respect to minimally invasive surgery. Kits may be provided that include any combination of rods or rails and optionally one or more modular plates. The kits may be refillable.

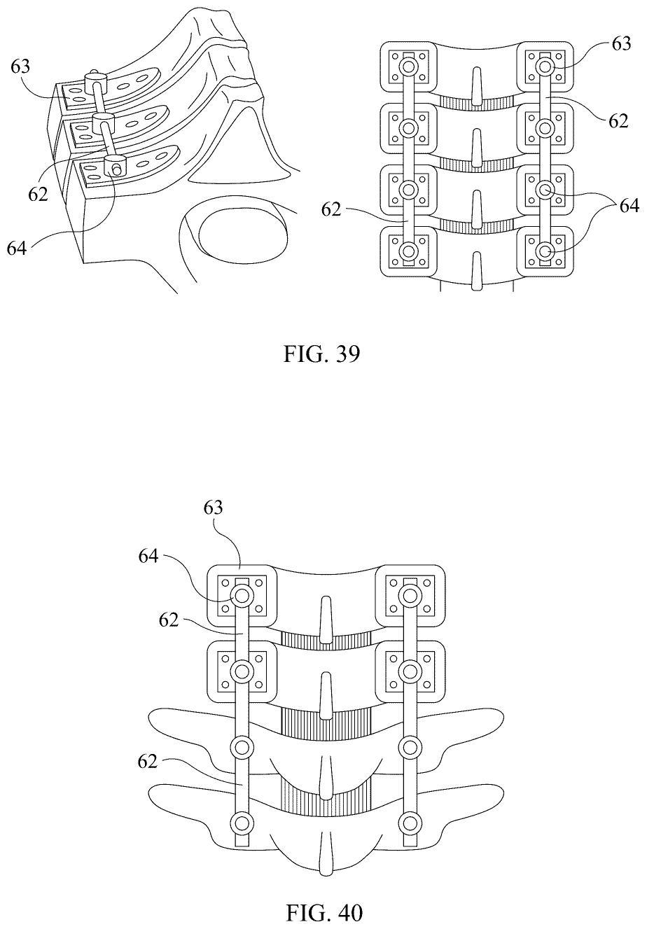

FIG. 39 is an example of multilevel cervical plate construct in accordance with non-limiting examples. This is an example of a cervical implant that includes multiple bone plates having polyaxial tulips with rods therebetween.

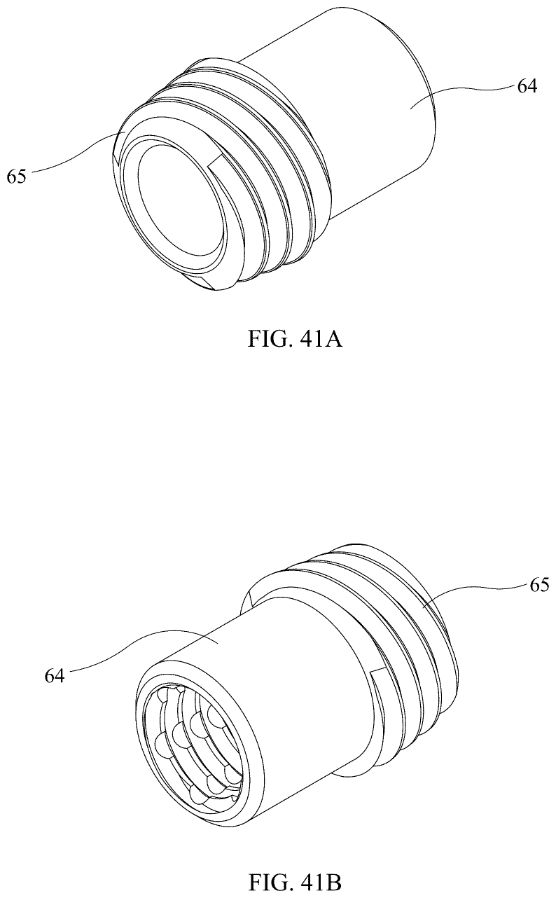

FIG. 40 depicts a hybrid cervicothoracic plate/pedicle screw construct.

In example embodiments, segmental locking plates with polyaxial or monoaxial saddles (tulips) may be used for internal fixation of extremity fracture. One, two or plurality of the plates with one, two or plurality of locking screws may be fitted with one, two or plurality of poly- or monoaxial tulips on either side of the fracture, The individual plates may be connected with one, two or plurality of rods or rails or combinations of such.

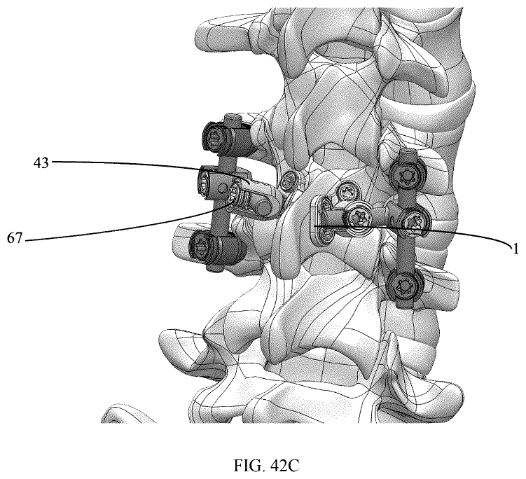

FIGS. 42A-42C depict a whole system in accordance with non-limiting example embodiments. In particular, FIGS. 42A-42C show a summary of example implants attached to a rod system to provide supplemental fixation. Example systems include multiple bone plates 1, screws and tulip screws 43 fixing the bone plates to the spine of a patient, and fixing the bone plate to a rod system via the tulip to provide supplemental fixation. The present systems may provide supplemental fixation to another system. For example, the present system may be a whole construct attached to a 5.5 rod system. A cap 67 may be inserted into the tulip of a tulip screw to hold a rod, plate, etc. within the tulip.

FIG. 42B depicts a frontal view and FIGS. 42A and 42C depict oblique images of the lumbar spine with a non-limiting example plating system in use as a supporting mechanism to a standard 5.5 pedicle screw rod systems. The standard pedicle screws are shown (4 blue tulips with standard 5.5 mm rods×2). The depicted example plating system includes three locking screws into the bone (grey), the tulip locking screw (yellow 43) and the locking plate itself 1. In the depicted example, the (purple) cross connector joins the plate 1 to provide supplemental fixation to the bilateral 5.5 pedicle screw rod system.

In example embodiments, the screw system can also be for 3.5, 4.0, 4.75, 6.0 and quarter inch diameter screw/rod system.

According to example embodiments the present systems may be removable from a patient with minimal to no damage, as the present screws may produce smaller holes (e.g. 4 mm holes) in a location that does not cause damage to the spine of the patient.

### Kits

Also provided are kits that include a bone plate or system as described herein and at least one additional component including instructions for use of the plate or system, or one or more accessories relating to the device or system, such as for example, screws, rods, rails, offset connectors, drill guides, guide wires, screwdrivers, counter torque, retaining pins, wires drills or other tools for insertion, placement, attachment or guidance of the device(s) or system(s) into a patient. Example embodiments include a screw corresponding to at least one of the screw openings, a screw-in attachment corresponding to at least one of the screw openings, a rod, a plate, a bar, a rail, another connector, instructions for use or insertion of the bone plate, and/or a tool for visualizing, applying or inserting the bone plate into a patient.

Further kits provided herein may include multiple bone plates of varying sizes and/or shapes. In example embodiments, each bone plate of the multiple bone plates may include a main body having a plurality of screw openings, in which the main body is configured in shape to correspond to bone of a patient, and in which at least one screw opening of the plurality of screw openings has threading configured to accept a respective screw or screw-in attachment therein. Example kits may also include at least one additional component including instructions for use of the multiple bone plates or a system including the multiple bone plates, or one or more accessories relating to the device or system, such as for example, screws, rods, rails, offset connectors, drill guides, guide wires, screwdrivers, counter torque, retaining pins, wires drills or other tools for insertion, placement, attachment or guidance of the device(s) or system(s) into a patient. Example embodiments include a screw corresponding to at least one of the screw openings, a screw-in attachment corresponding to at least one of the screw openings, a rod, a plate, a bar, a rail, another connector, instructions for use or insertion of the bone plate, and/or a tool for visualizing, applying or inserting the bone plate into a patient.

In example kits, the screw may be selected from one or more of a lag screw, a lock screw, and a tulip screw such as a monoaxial tulip screw or a polyaxial tulip screw.

### Methods

Also provided are methods that include placing a bone plate at a desired location in a patient in need of the bone plate, for example for stabilization or fixation, and securing the bone plate to bone of the patient by screwing at least one screw through a corresponding screw opening of the bone plate, into the bone of the patient. In example embodiments, the bone plate includes a main body having a plurality of screw openings therein, the main body of the bone plate is shaped to correspond to bone of the patient at the desired location to which the bone plate is to be applied, and the at least one screw opening of the plurality of screw openings has threading configured to accept a respective screw or screw-in attachment therein.

In example methods, there is a tulip attaching to the bone plate that is configured to accept a rod to attach to another plate or tulip screw.

Example methods may further include adding at least one screw-in attachment to a corresponding screw opening. The at least one screw-in attachment includes a screw end configured to be screwed into a corresponding screw opening of the bone plate, and an attachment end configured to attach a support device thereto. The support device may include a further plate, screw, rod, bar, rail or other connector. The attachment end may include a tulip configured to receive a support device.

According to example embodiments, in example methods, the main body may be bent and have a first side and a second side at an offset angle with respect to the first side. The first side may include at least one screw opening and the second side may define at least one screw opening.

According to example embodiments, methods provided herein may include open surgery methods or minimally invasive surgery (MIS) methods to be performed on a patient.

In example embodiments, the present application provides MIS methods for placement of MIS plates into a patient.

FIG. 30 depict a bone plate 25, such as a spinal lamina plate that can be adapted for minimally invasive surgery (MIS) insertion, in accordance with non-limiting example embodiments. FIG. 30 depicts plates 25 of different shapes with different configurations of holes 27, such as threaded holes for plate holders or drill guides. The MIS spinal lamina plates may also include at least one band or endoscopic suture 37 that can be passed arthroscopically, an anchor 62, a drill guide 45 (which may be removable), and/or spikes 29 for initial stability of the plate with respect to bone to which the plate is to be applied.

FIG. 31 shows use of a locking screw 46 that accommodates bands for additional spine fixation and a locking cap 50. Such bands can be passed arthroscopically endoscopically. FIG. 31 depicts example embodiments in which a locking screw 46 or cap 50 within the inside channel is visualized. FiberWire 51 or any other synthetic material suture, tape or band can be passed through the inside channel in the screw. There may be one or more groves in the inside of the screw 46 for the Fiber Wire or other material. This can be used to augment soft tissue or bone repair. For instance, it can be used for endoscopic pars or other fracture repair. The locking caps 50 may be threaded as well. The inner part of the screw may have one, two, three, or more grooves for the suture strand to sit on to prevent cutting of the strand by the locking cap. The locking screw can be passed for example through an inside channel 49 in the screw. The locking screw may include a lower screw portion 47 and an upper locking screw portion 48.

The spine locking plate will have one, two or more holes for the screws or threaded knobs. Threaded knobs will have threads matching the locking threads inside of the plate. However it will not protrude beyond the plate itself. Example screws on the contrary, will have both locking threads that will lock into the threaded portion on the plate, but there will be a shaft protruding past the plate as well. The shaft may have partial threads, complete threads or no threads at all. The tip of such screw can be blunt, self-drilling or self-tapping. Such screw can be hollow or cannulated. It can also be solid. A combination of such screws can be used in any given plate. A plate may be straight, curved, bent or have any other complex shape to accommodate the variable spinal anatomy.

Also provided are methods that include placing a bone plate system having a bone plate and at least one drill guide, at a desired location in a patient in need thereof, creating a pilot hole in the bone of the patient through at least one drill guide, removing the drill guide from the bone plate system, and screwing a screw into the pilot hole in the bone of the patient through the bone plate to attach the bone plate to the bone of the patient. In example embodiments, the bone plate includes a main body having a plurality of screw openings therein, in which the main body is shaped to correspond to bone of the patient to which the bone plate is to be applied, and at least one screw opening of the plurality of screw openings has threading configured to accept the screw from the at least one screw therein. In example embodiments, the at least one drill guide is positioned on the bone plate at a corresponding screw opening of the plurality of screw openings.

In example methods, creating the pilot hole may be performed by drilling in bone of the patient through the at least one drill guide or by using an awl to create the pilot hole.

Further methods provided herein include the following:
a) determining a size of a lamina of a patient,
b) selecting locking plate of a corresponding size to be applied to the patient, based on the determined size and shape of the lamina,
c) attaching a plate holder/drill guide into a preloaded drill guide on the selected locking plate,
d) placing the locking plate over the lamina of the patient,
e) creating a first pilot hole in the lamina of the patient through a first hole in the locking plate using a first preloaded drill guide using a drill or an awl corresponding to the first hole in the locking plate,
f) screwing a first locking screw in the first pilot hole in the lamina of the patient through the first hole in the locking plate,
g) repeating steps e) and f) at least one time, creating a second or more pilot hole by drilling or awling a second or more pilot hole through a second or more hole in the locking plate, and screwing a second or more locking screw in a corresponding pilot hole in the lamina of the patient through a corresponding hole in the locking plate, and
h) removing the plate holder from the patient.

Example methods may further include the following steps:
i) placing a tulip screw into a tulip screw hole in the locking plate, and
j) placing an offset connector into a tulip portion of the tulip screw to connect to a previously placed pedicle screw construct to the locking plate.

In other example embodiments, methods may include:
i) placing a tulip screw into a tulip screw hole in the locking plate and
j) placing a rod or rail in a tulip portion of the tulip screw to connect the locking plate to another plate anchor or pedicle screws.

In example methods the locking screw tulip may accept a rail or a rod, such as a 5.5 rod.

In example methods, the plate holder may be a spring loaded or fixed awl having a spring and the method further includes tapping the spring loaded to anchor the locking plate.

In example embodiments, determining the size of the lamina may be performed using a template.

A pilot hole, such as the first pilot hole and subsequent pilot holes, may be created in the lamina of the patient may be created by drilling or by malleting or pushing an awl. Example locking plate systems and methods provided herein may use using awls and/or by drilling, using e.g., small depth drills with safety guides/stops to create central pilot holes in bone, which may be necessary or desired to obtain the appropriate screw trajectory for the locking system to work. Drilling or creating a pilot hole may be necessary to the depth of 3 to 6 mm. The thickness of the plate may be for example 2 mm to 6 mm, but most commonly 2.5-4.5 mm, which may need to be added to the drilling depth.

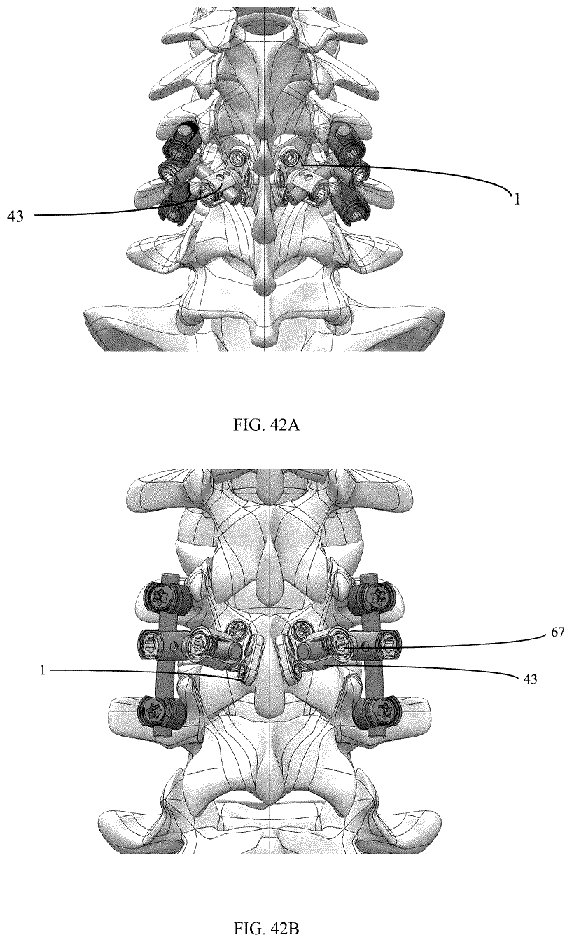

Screw-in guides or drill guides, for example as depicted in FIGS. 41A and 41B may be preloaded onto bone plate or they can be threaded at a later stage. In examples, screw-in drill guides may already be fixed to the bone plate when given to the end user, to save time and effort during implantation of the bone plate.

FIGS. 41A and 41B are images of the screw-in drill guides which will be inserted into the plate. They can be removed with a standard screw driver placed in the set. The guides are required to center the pilot hole via awl or drill guide. This guides are then removed with the driver and the screw is inserted. The guides center the screw trajectory and allows the locking mechanism to screw into the plate.

According to example methods, a screw end 65 of the screw-in guide is screwed in to a screw hole of the bone plate. After a pilot hole is made in bone of a patient (e.g. by applying a tool such as an awl or drill through the guide portion 64 of the screw-in guide), the screw in drive may be unscrewed and removed from the system and patient and a screw may be thereafter drilled into the same drill hole, utilizing the pilot hole to assist with alignment of the screw being screwed into the screw hole and penetrating into bone under the screw hole.

Methods and systems provided herein may also include methods and systems of using the present bone plates to revise a loose screw without removing an old construct. The present locking plates are capable of connecting to any prior pedicle screw system via offset cross-connectors. This universal compatibility of a segmental spine anchor is unique.

The bone plate, such as a spinolaminar plate can be used in the minimally invasive or open fashion to revise a loose screw, such as seen in cases of nonunion, hardware loosening or proximal junctional failure. In this application the old construct can be retained and the bone plate 25 can be placed onto the spine and attached to the existing construct using a lateralizing, offset or iliac connector 63.

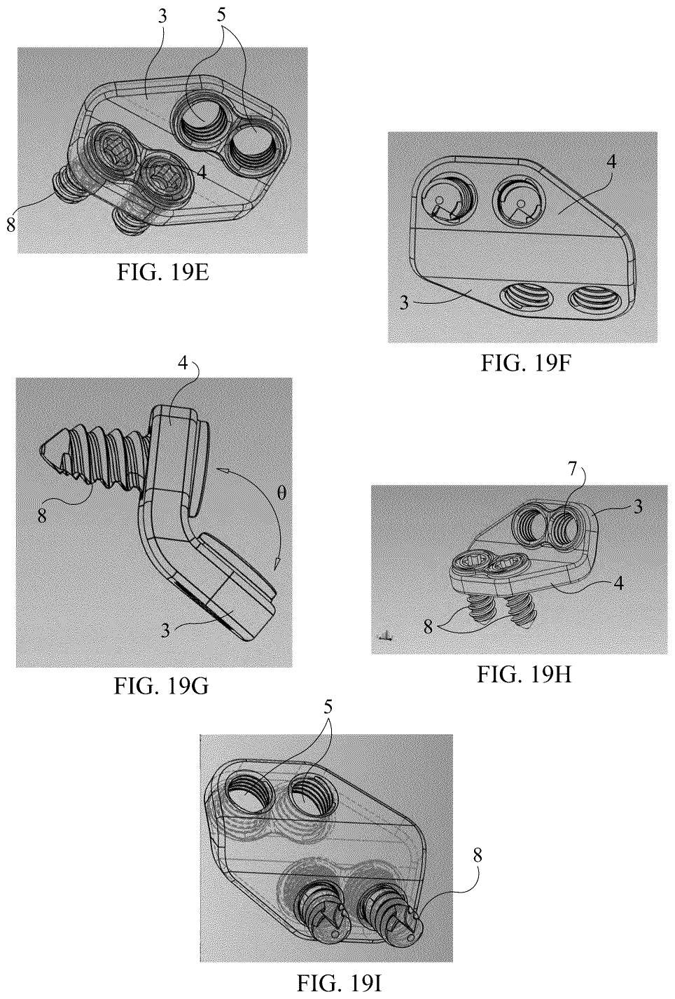

FIG. 26 shows a 3-hole plate 25 for pedicle screw augmentation according to non-limiting example embodiments, and also lateralizing offset connector. This demonstrates using spine plate for supplemental fixation around pedicle screw construct. A straight or curved offset lateralizing connector is used to connect the tulip 43 on the plate 25 to a rod 44. The plate 25 may be fixed to bone of a patient using one or more screws 31, which as shown in FIG. 26 may be for example at a diverging (or converging) angle with respect to each other.

The example bone plate may have one, two, three or more locking, non-locking or hybrid holes that may be configured to accommodate one or more screws, and/or one or more screw-in attachments, such as the example depicted tulip screw, which may be adapted to attach to a rod or connector.

Also provided are patient anatomy-specific spinolaminar locking plates and fixation systems for segmental fixation, for example, during spine fusion surgery. Such methods include measuring and/or visualizing regional bone density in a patient, prior to selecting a suitable bone plate from the present bone plates and bone plate systems. These methods may be used for example for spinolaminar locking plate segmental fixation of the spine.

According to example methods of the present application, patient specific methods are provided of using regional bone density to guide optimal segmental spine fixation. According to non-limiting examples, a Densitometry Map may be used to determine preferred placement and type of plate and system to be used in a particular patient. The CT scan is used to measure regional bone density (in HU—Houndsfeld units—available on most of the PACS and DICOM viewing software). The regions of interest for taking CT scans are spinolaminar junction, lamina, pars, facet, body and pedicle. The regional bone density of a patient is recorded for the key posterior elements (lamina, pars, spinous process, superior articular process, inferior articular process, pedicle), pedicles and vertebral body for each side. Alternatively, this can be automated on PACS and different colors/shading may be assigned to different HU values to create a Density Map or DensoMap. Therefore, in accordance with non-limiting methods of the present application, a Densitometry Map is obtained of a patient prior to surgery. Based on the CT results and Densitometry map, a provider selects which plate(s) to use in which location on a patient. It is contemplated that selection of the appropriate plate(s) may be assisted by use of a computer or AI.

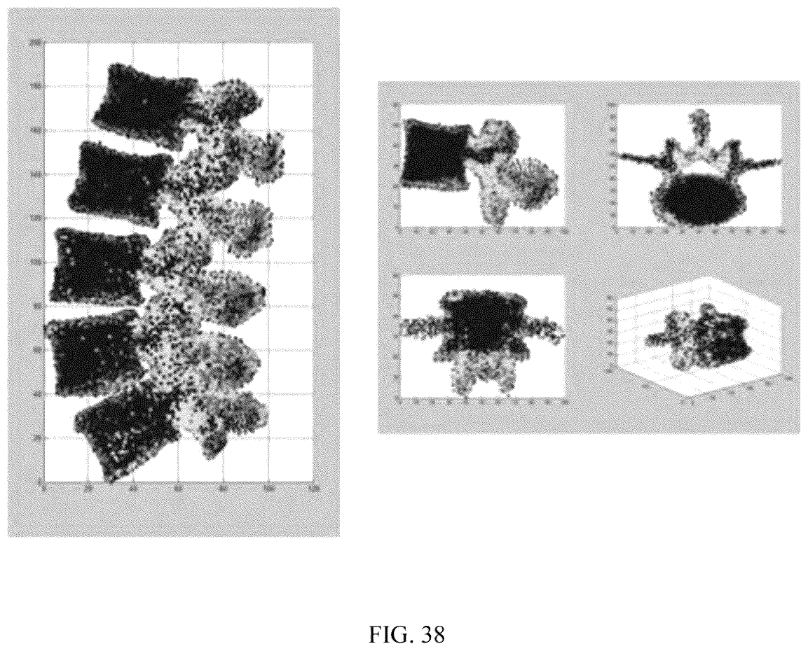

FIG. 38 depicts a Densitometry Map, which may be used to measure and or visualize regional bone density in a patient, in accordance with non-limiting example embodiments of the present application, in patient-specific methods of spinolaminar locking plate segmental fixation of the spine. In the figure, the dot color correlates to bone quality, with lighter or warmer areas corresponding to higher quality bone.

Depending on the regional bone density, as determined by the Density Map/CT scan, a spinolaminar plate with an enhanced screw cluster in the region of less dense bone may be utilized. For instance, if the HU measurement of the spinolaminar junction is less than 100 indicating osteoporosis, then a plate with more screw holes in the midline (at the spinolaminar junction may be used). If the HU measurement is the lowest at the facet, then a spinolaminar plate with an extended screw cluster at the facet (i.e. more holes laterally) may be selected.

In example methods, a preoperative bone density measurement can be used to plan posterior element fixation in the spine, especially in osteoporotic bone. custom plates may be developed using the CT and densitometry. custom plates can be designed after looking at densitometry and ct scans; the outrigger can by custom designed to match the patient's anatomy allowing the user to insert longer screw s into pedicle improving strength.

The present novel plating system can be used for supplemental fixation in osteoporosis, or weak bone, it can be used when a pedicle breaks or is dysplastic as a salvage point of fixation, and it can ultimately be used standalone to provide posterior non-segmental and segmental stabilization of the spine. The present locking plates provide a segmental anchor, attaching to one vertebra, as opposed to older plates that bridge several segments (vertebrae) together.

The present segmental locking plates are the next step in evolution of spinal segmental fixation, following wires, hooks and pedicle screws. Every two to three decades there has been a new segmental anchor for the spine, with the subsequent designs incorporating and combining prior ones.

In example embodiments, plates can be placed at the same level and same side as pedicle screws since they rely on different sections of vertebrae. In example embodiments, plates can be combined with pedicle screws and hooks in the same construct. Plates can be also used for spine derotation/compression/distraction during scoliosis/kyphosis reduction maneuvers which would avoid the risk of pedicle screws breaking into the spinal canal during corrective maneuvers.

The present application provides locking plates with tulips that can also be used for extremity fracture fixation in either open or minimally invasive fashion. This is unique as no prior implants or fixation systems could be utilized for both spine or extremity fixation.

The present locking plates provide an ability to place a pedicle screw using cortical trajectory (medial to lateral) as well as extrapedicular screws (lamina, facet, pars, spinous process). The ability to do both with the same anchor is unique.

The present plates create a fixed angle construct in osteoporotic bone which obviates any need to use cement augmentation of pedicle screws, decreasing costs and improving safety.

Level-specific plates may be provided according to non-limiting example embodiments. The plates may be offered, for example, in standard version for stronger bone (HU more than 100-120) in small, medium and large sizes for Left and Right sides. There will be an augmented lateral or facet version with more screw holes laterally to engage the facet or allow for cortical trajectory pedicle screw placement. This will also be offered in small, medium and large for both Left and Right sides (FA or facet augmented plates). There will also be augmented medial plates for patients with less dense spinous processes and laminae (SL or spinolaminar augmented plates). Those will also be offered in small, medium, and large both for the Left and Right sides.

Accordingly, the present application may provide systems, sets or kits of level-specific plates that may be provided or sold for example as entire kits, and/or as refills. For example, there may be kits that include standard plates in small, medium and large sizes (and/or optionally XS, XL or other sizes). There may be kits that include Augmented Lateral or Facet (FA) plates that include left plates in S, M, L and optionally other sizes, and right plates in S, M, L and optionally other sizes. There may be kits that include Augmented Medial plates that include left plates in S, M, L and optionally other sizes, and right plates in S, M, L and optionally other sizes. Example kits may also include combinations of e.g., all three types of plates (standard, augmented lateral or facet and augmented medial plates), or any combination thereof. Kits that include any of the present plates may also include for example hardware and/or tools for use with the plates.

According to example embodiments, the present plates may be configured such that they can be used with or coupled to existing screws or rods, for example, produced by different manufacturers. The anchoring plate includes multiple through-holes (or screw holes) for receiving bone screws to secure the anchoring plate to multiple structures. In non-limiting example embodiments, the through-holes can be threaded so as to engage the threads of the shank and further restrict the range of angles at which the bone screw penetrates and seats within the structure, increasing the precision of the trajectory of the bone screw as it seats within the bone.

Optionally, one or more of the bone screws can be a locking screw that includes a threaded head/upper shaft or shank to resist backing out of a seated position in the through-holes. The threads of the shank and screw head may engage complementary threads of the through-hole. A portion of the through-hole may or may not include an expanded diameter that complements finely pitched threads of the head/upper part of the shank of the locking screw. As the locking screw seats within the bone of the lamina, the threads of the head/upper shank or shaft engage the threads of the expanded diameter of the through-hole so that the locking screw resists backing out of the bone. The locking screw can be particularly useful where the bone stock at an implantation site has degenerated, deteriorated, deformed or is otherwise of poor quality.

According to non-limiting example embodiments, the anchoring plate may be made of titanium or titanium alloy. However, other biocompatible materials such as stainless steel, cobalt-chrome alloy and/or PEEK and/or other polymers can be used. As will be appreciated upon reflecting on the different embodiments, the structures described herein can vary in size and shape based on factors such as material of construction, anatomical structure of the implantation site, implantation technique and targeted system performance (e.g., stiffness).

The angle of orientation of the shanks of the different bone screws relative to the sagittal plane vary and/or the range of angles of orientation of the shanks of the first, second, and third screws relative to the sagittal plane do not overlap. The splayed or converging arrangement of the bone screw shanks provides improved resistance to pull-out force, thereby, reducing or eliminating the need to notch or remove portions of the bone and/or provide additional hooks or grafted structures.

Example level-specific plates in accordance with the present application, may be provided in different sizes and shapes with different configurations of screw hole clusters, which may be selected depending on the regional bone density of a patient.

Non-limiting example embodiments of the present application may include plates having clusters of screw holes that are positioned to optimize fixation in a patient when the plate is inserted and screwed into a patient using screws provided herein and known to those skilled in the art, in particular at locations with low bone density.

According to non-limiting example embodiments, the same steps may be used for the additional levels as well. That is, it is possible that one level of a patient will require a standard plate on one side, a facet augmented plate on the other side, spinolaminar augmented plates on both sides at another level, etc. Accordingly, by using the present methods of performing a CT or bone density scan of a patient to determine the bone density at a particular location, a practitioner may select the type (e.g., size of screw holes and how many screw holes clustered in which location), and size of plate, as well as placement. This will ensure customized fixation and optimized screw purchase that is region- and level-specific for a given patient. This will potentially minimize the screw pullout, proximal and distal junctional failure and the need for revision surgery. Kits in accordance with the present application may provide the various types and sizes of plates to be used.

In extreme cases of severe osteoporosis (HU less than 70-80), as determined by the CT scan of the present methods, and prior compression fractures, spinolaminar plates can be used in accordance with the present application for sublaminar fixation, which may accommodate an eyelet for a sublaminar band or wire to augment the locking screws. In cases where conventional rather than locking screws are preferred, a blocking (anti-screw backout) plate may be utilized to prevent screw backout. Accordingly, methods, devices and kits of the present application may include plates that may accommodate eyelets, sublaminar bands or wires, locking screws, non-locking screws, locking posts (i.e. smooth shank device with limited threads closer to the head), a blocking plate, etc.

For severely osteoporotic bone (HU less than 70-80), as determined by a CT scan in accordance with example methods herein, a hybrid construct may be selected for example, with a pedicle screw used at the same level and same side as the plate with plate and screw connected via a lateralizing or iliac-type offset connector, as described herein.

Plates in accordance with example embodiments may also accommodate non-locking or conventional screws for a more dynamic construct. In this case, a secondary blocking plate with the polyaxial or monoaxial tulips might be utilized to prevent the screws backing out. This combination will create a blocking (anti-backout) as opposed to a locking (fixed angle) construct.

The present customized level-specific bone density-specific and patient specific segmental fixation utilizing rods of different material (cobalt chrome, stainless steel, titanium alloy and pure commercial titanium or other metals, materials, shapes and allows), and different segmental anchors (pedicle screws, spinolaminar plates, sublaminar bands, wires and hooks) will optimize the construct strength, possibly improve maintenance of correction, and minimize junctional failure.

As more spinal fusion surgery has been done, the prevalence of adjacent level disease has risen and will continue to rise. The bone plate systems of the present application will allow for attaching to multiple rod-screw constructs above and/or below fusions to simplify the surgery itself. By clamping onto the rod and obtaining fixation to the bone, this will avoid having to open the entire wound and remove all the caps/rods when performing revision fusion surgery for adjacent level disease. This will reduce the morbidity, risk of infection, muscle dissection, soft tissue trauma and reduce the amount of time to perform a revision surgery above or below a previous fusion that has developed adjacent level disease. The spinolaminar system will also be compatible with multiple companies' domino connectors and rod systems to simplify and broadening its applications.

While the present disclosure has been described in terms of exemplary aspects, those skilled in the art will recognize that the present disclosure can be practiced with modifications and/or variations within the spirit and scope of the application. The examples provided herein are merely illustrative and are not meant to be an exhaustive list of all possible designs, aspects, applications or modifications of the present disclosure

We claim:

1. A bone plate comprising
a main body having a plurality of screw openings,
wherein the main body is configured in shape and size to correspond to bone of a patient to which the bone plate is to be applied, and
wherein a first screw opening of the plurality of screw openings has threading configured to accept a respective screw or screw-in attachment therein.

2. The bone plate of claim 1, wherein the first screw opening of the plurality of screw openings is at an oblique angle with respect to a second screw opening of the plurality of screw openings, such that a first screw or screw-in attachment inserted into the first screw opening is at an oblique angle with respect to a second screw or screw in attachment inserted into the second screw opening.

3. The bone plate of claim 1,
further comprising an offshoot section from the main body, the offshoot section defining an offshoot screw opening that has threading configured to accept an offshoot screw or screw-in attachment therein.

4. The bone plate of claim 1,
further comprising at least one tulip on a top surface of the main body as an integrated part of the bone plate.

5. The bone plate of claim 1, wherein at least one surface of the main body is roughened for bone integration.

6. The bone plate of claim 1, wherein the bone plate further includes a plurality of spikes for initial fixation to the bone.

7. A bone plate system comprising
a bone plate according to claim 1, and
at least one screw or screw-in attachment corresponding to a respective screw opening of the plurality of screw openings.

8. The bone plate system of claim 7,
wherein the screw-in attachment comprises a locking eyelet having threads for screwing the locking eyelet into a screw opening of the plurality of screw openings, and having an eyelet opening configured in size and shape to allow a band, wire or cable to pass therethrough.

9. The bone plate system of claim 7,
wherein the screw-in attachment comprises a blocking plate having
a protrusion or screw on a bottom of the blocking plate, the protrusion or screw being attachable to an opening defined on a top side of the bone plate, and
an attachment mechanism on a top side of the blocking plate.

10. The bone plate system of claim 9, wherein the attachment mechanism is a tulip.

11. The bone plate system of claim 10, wherein the tulip is a polyaxial tulip or a monoaxial tulip.

12. The bone plate system of claim 7, wherein the screw comprises at least one screw selected from the group consisting of a lag screw, a lock screw, and a tulip screw.

13. The bone plate system of claim 7, further comprising a drill guide corresponding to at least one of the at least one screw opening.

14. The bone plate system of claim 12, wherein the drill guide is preloaded onto the bone plate at a corresponding screw opening.

15. The bone plate system of claim 7, wherein the bone plate further comprises at least one tulip on a top surface of the main body as an integrated part of the bone plate.

16. A method comprising
placing a bone plate at a desired location in a patient in need thereof, wherein the bone plate comprises:
a main body having a plurality of screw openings therein,
wherein the main body is shaped to correspond to bone of the patient at the desired location in the patient to which the bone plate is to be applied, and
wherein at least one screw opening of the plurality of screw openings has threading configured to accept a respective screw or screw-in attachment therein, and
securing the bone plate to the bone of the patient by screwing at least one screw through a corresponding screw opening of the plurality of screw openings, into the bone of the patient.

17. The method of claim 16, further comprising adding at least one screw-in attachment to a corresponding screw opening.

18. The method of claim 16, wherein the support device comprises a further plate, screw, or rod.

19. The method of claim 16, further comprising
placing a tulip screw into a tulip screw hole in the bone plate, and
placing a rod in a tulip portion of the tulip screw to connect the bone plate to another plate anchor or pedicle screws.

20. A kit comprising
the bone plate of claim 1, and
at least one additional component selected from the group consisting of instructions for use or insertion of the bone plate or system, one or more screws, screw-in attachments, rods, plates, rails, bars, offset connectors, drill guides, guide wires, screwdrivers, counter torque, retaining pins, wires drills and other tools for insertion, placement, attachment or visualization of the device(s) or system(s) into a patient.
